(19) United States
(12) Patent Application Publication
(10) Pub. No.: US 2025/0186674 A1
(43) Pub. Date: Jun. 12, 2025

(54) MANUAL ACTUATION OF ROBOTIC SUCTION IRRIGATORS

(71) Applicant: CILAG GMBH INTERNATIONAL, Zug (CH)

(72) Inventors: Kris E. KALLENBERGER, Cincinnati, OH (US); Disha V. ESTERA, Cincinnati, OH (US); David C. GROENE, Cincinnati, OH (US); Eric STOUT, Cincinnati, OH (US); Laura A. NYE, Cincinnati, OH (US); Rudolph H. NOBIS, Mason, OH (US)

(73) Assignee: CILAG GMBH INTERNATIONAL, Zug (CH)

(21) Appl. No.: 18/531,502

(22) Filed: Dec. 6, 2023

(51) Int. Cl. A61M 1/00; A61B 34/30

(52) U.S. Cl. CPC A61M 1/774; A61B 34/30; A61M 1/7413; A61B 2034/302; A61B 2217/005; A61B 2217/007

(57) ABSTRACT

A suction irrigator includes a drive housing, a flow control system arranged within the drive housing and including a flow manifold that houses an internal valve, and a drive input rotatably mounted to the drive housing. A capstan assembly is arranged within the drive housing and is operatively coupled to the flow manifold such that rotation of the drive input actuates the capstan assembly and thereby operates the internal valve between closed and open positions. A button is mounted to the drive housing and operatively coupled to the flow manifold such that manually depressing the button operates the internal valve between the closed and open positions. A decoupling mechanism mechanically decouples the button from the capstan assembly when the button is manually pressed such that manual movement of the button fails to backdrive the drive input or a motor operable to drive the drive input.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

One type of MIS instrument is a laparoscopic suction/irrigation tool or system, commonly referred to as a “suction irrigator”. Suction irrigators are specialized instruments that allow laparoscopic surgeons to irrigate, clean, and aspirate a surgical site during a procedure. The system generally consists of a tube, a suction pump, and a fluid pump. The tube is inserted into the surgical site through one of the small incisions, and the suction pump removes any fluids, debris, or blood that accumulates during the surgery. A fluid pump or IV bag is able to deliver a sterile saline solution to the surgical site to keep it clean and free of debris.

In robotic surgery, the suction irrigator is typically mounted to a robotic tool driver, and a user (e.g., a surgeon) is able to remotely operate the suction irrigator by grasping and manipulating one or more controllers that communicate with the tool driver. The user inputs are processed by a computer system incorporated into the robotic surgical system, and the tool driver responds by actuating the suction irrigator as directed.

Suction irrigators on robotic platforms do not typically allow for on-robot manual actuation, which could backdrive the drive inputs and the damage the motors used to actuate the drive inputs. Nevertheless, manual actuation of suction irrigators while “on-robot” may be desired at times, but should be accomplished without back driving the motors of the robotic tool driver or interfering with the controls of the robot.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIG. 4 is a bottom view of the drive housing of FIG. 2, according to one or more embodiments.

FIGS. 5A and 5B are exposed isometric views of the interior of the drive housing of FIG. 2 taken from left and right vantage points, respectively, according to one or more embodiments.

FIGS. 5C-5E depict an example decoupling mechanism that may be incorporated into the flow control system of FIGS. 5A-5B.

FIGS. 6A and 6B are exposed isometric views of an example flow control system, according to one or more embodiments of the present disclosure.

FIG. 7A is an isometric view of another example flow control system, according to one or more additional embodiments of the present disclosure.

FIG. 7B is an exploded view of the capstan shaft and the cam lifter of FIG. 7A, according to one or more embodiments.

FIGS. 8A and 8B are exposed isometric views of another example flow control system, according to one or more additional embodiments of the present disclosure.

FIG. 9 is an enlarged, isometric view of another example flow control system, according to one or more additional embodiments of the present disclosure.

FIG. 10 is an enlarged, isometric view of another example flow control system, according to one or more additional embodiments of the present disclosure.

FIGS. 11A and 11B are exposed, isometric views of another example flow control system, according to one or more additional embodiments of the present disclosure.

FIG. 12 is an isometric view of another example flow control system, according to one or more additional embodiments of the present disclosure.

FIG. 13 is an isometric view of another example flow control system, according to one or more additional embodiments of the present disclosure.

FIG. 14 is an isometric view of another example flow control system, according to one or more additional embodiments of the present disclosure.

FIG. 15 is an isometric view of another example flow control system, according to one or more additional embodiments of the present disclosure.

FIG. 16 is an isometric view of another example flow control system, according to one or more additional embodiments of the present disclosure.

FIGS. 17A and 17B are left and right isometric views, respectively, of another example flow control system, according to one or more additional embodiments of the present disclosure.

FIG. 18 is an exposed, isometric view of yet another flow control system 1800, according to one or more additional embodiments of the present disclosure.

FIGS. 19A and 19B are top and isometric views, respectively, of another example flow control system, according to one or more additional embodiments of the present disclosure.

FIGS. 20A-20C are exposed top, side, and isometric views, respectively, of another example flow control system, according to one or more additional embodiments of the present disclosure.

FIGS. 21A and 21B are alternative capstan assemblies that may be used in conjunction with the flow control system of FIGS. 20A-20C.

FIG. 22 is an exposed isometric view of another example flow control system, according to one or more additional embodiments of the present disclosure.

### DETAILED DESCRIPTION

The present disclosure is related to robotic surgical systems and, more particularly, to suction irrigators with manual irrigation and suction buttons that are decoupled from the robotic drive motors that operate the flow control system of the suction irrigator.

The flow control systems described herein include modifications that decouple manual irrigation and suction buttons from the capstan assemblies used to selectively actuate the flow control systems. Various enabling and decoupling mechanisms may be incorporated into the flow control systems described herein to allow the irrigation and suction buttons to be manually pressed (actuated) while the suction irrigator is mounted to the robotic tool driver (i.e., “on-robot”), but without back driving the motors of the robot or interfering with the controls of the robot. This may prove advantageous in allowing bedside assist to manually actuate the robotic suction irrigator while on-robot without risk of damaging drive motors. Moreover, while off-robot, the decoupled motion of the manual irrigation and suction buttons does not rotate the robotic drive inputs against the hand of the user if being actuated manually. Furthermore, when manually actuating the irrigation and suction buttons, much of the force due to friction in the robotic drive system is eliminated allowing for lower manual actuation forces relative to a coupled robotic suction irrigator.

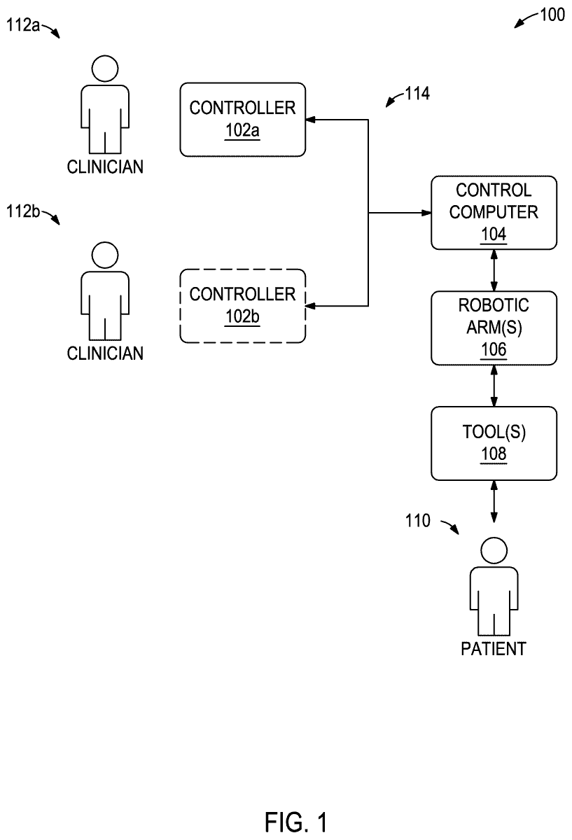

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

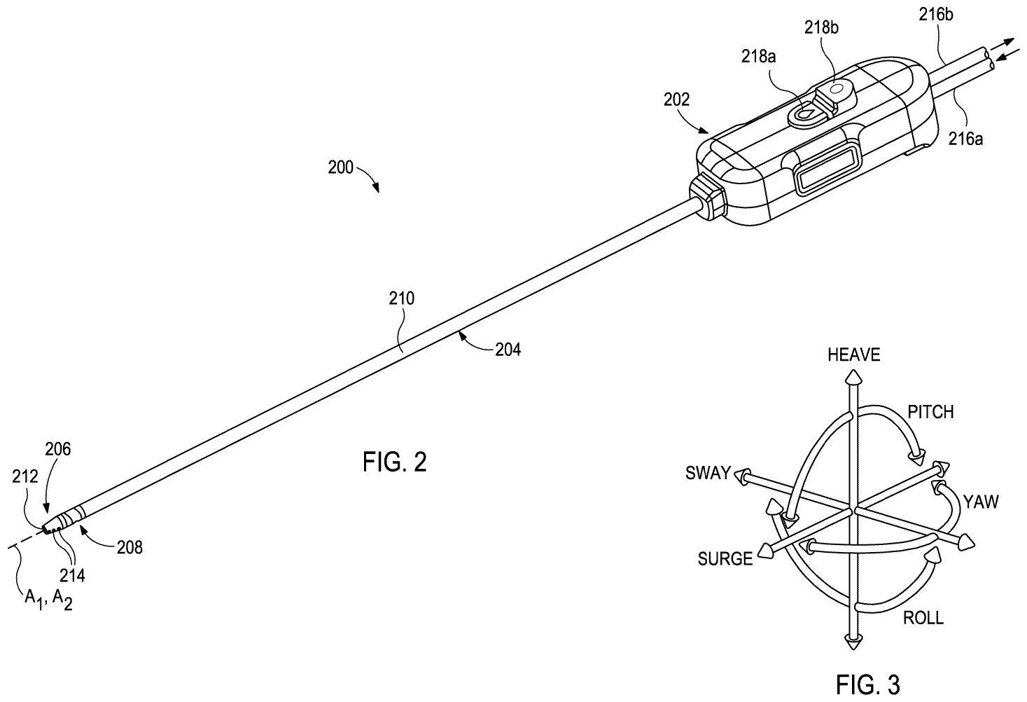

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. Accordingly, the surgical tool 200 may be designed to be releasably coupled to a tool driver included in the robotic surgical system 100. In other embodiments, however, aspects of the surgical tool 200 may be adapted for use in a manual or hand-operated manner, without departing from the scope of the disclosure.

As illustrated, the surgical tool 200 includes a drive housing 202 and an elongated shaft 204 that extends from the drive housing 202 and terminates at a distal tip 206. In at least one embodiment, the surgical tool 200 may further include a wrist 208 (alternately referred to as a “wrist joint” or an “articulable wrist joint”) that interposes (couples) the distal tip 206 and the distal end of the shaft 204. In applications where the surgical tool 200 is used in conjunction with a robotic surgical system (e.g., the robotic surgical system 100 of FIG. 1), the drive housing 202 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the drive housing 202) to a robotic manipulator. The term “proximal” refers to the position of an element closer to the robotic manipulator and the term “distal” refers to the position of an element closer to the distal tip 206 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the distal tip 206 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

In the illustrated embodiment, the surgical tool 200 comprises a suction irrigator configured to irrigate and clean a surgical site during operation. Accordingly, the surgical tool 200 will be referred to herein as the “suction irrigator 200”. The shaft 204 may be a generally hollow tube capable of conveying fluids along its entire length in either direction. As used herein, the term “fluid” refers to any liquid or gas, but the shaft 204 may also be configured to convey a vacuum along its length. The shaft 204 may include an outer tube 210, which houses an inner tube (not visible) configured to convey the fluids (or the vacuum) along the length of the shaft 204 (in either direction). The fluid may be conveyed within the inner tube of the shaft 204 with or without the conveyance of solids. The distal tip 206 may be operatively or fluidly coupled to one or both of the outer tube 210 and the inner tube.

The distal tip 206 may define an opening 212 configured to discharge or receive fluids. In some applications, for example, a fluid (e.g., water, a sterile saline solution, a pressurized gas, etc.) may be conveyed through the shaft 204 and discharged from the distal tip 206 to a surgical site via the opening 212. In other applications, however, fluids (e.g., blood, water, etc.) may be drawn into the shaft 204 via the opening 212 at the distal tip 206. In some embodiments, the distal tip 206 may provide or define one or more smaller openings 214 around its circumference to further allow fluids to flow laterally into and out the shaft 204.

The drive housing 202 may house a flow control system (not visible) operable to control the flow of fluids (including any solids that may be transported by the fluids) along the shaft 204 and between a surgical site and the shaft 204, or vice versa. The flow control system may include one or more valves in fluid communication with the fluid conduit (inner tube) forming part of the shaft 204, and actuation of the valves enables or prohibits the flow of fluids through the shaft 204 in either direction. The flow control system may further be in fluid communication with one or more hoses or conduits extending from the drive housing 202. In the illustrated embodiment, for example, the suction irrigator 200 includes a first conduit 216a and a second conduit 216b. The first conduit 216a may be configured to convey a liquid (e.g., water, a saline solution, etc.) to the suction irrigator 200 to be discharged from the shaft 204 at the distal tip 206 for irrigation purposes, and the second conduit 216b may draw a vacuum and may otherwise be configured draw fluids (e.g., blood, water, air, etc.) through the shaft 204 from the surgical site.

While not shown, in some embodiments, the suction irrigator 200 may further include a third conduit configured to convey a compressed gas (e.g., air, nitrogen, CO₂, etc.) to the suction irrigator 200 to be discharged from the shaft 204 at the distal tip 206 for aspirating. Alternatively, operation of the either the first or second conduits 216a,b may be reversed or otherwise configured to convey compressed gas to the suction irrigator 200.

In at least one embodiment, the distal tip 206 may be configured to move (pivot) relative to the shaft 204 at the wrist 208 to position the distal tip 206 at desired orientations and locations relative to a surgical site. To accomplish this, the drive housing 202 may include (contain) various drive inputs and mechanisms (e.g., gears, actuators, etc.) designed to control articulation of the wrist 208, and thereby control the angular orientation of the distal tip 206.

FIG. 3 illustrates the potential degrees of freedom in which the wrist 208 may be able to articulate (pivot) and thereby move the distal tip 206. The wrist 208 can have any of a variety of configurations. In general, the wrist 208 comprises a joint configured to allow pivoting movement of the distal tip 206 relative to the shaft 204. The degrees of freedom of the wrist 208 are represented by three translational variables (i.e., surge, heave, and sway), and by three rotational variables (i.e., Euler angles or roll, pitch, and yaw). The translational and rotational variables describe the position and orientation of the distal tip 206 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 208 (e.g., X-axis), yaw movement about a second axis of the wrist 208 (e.g., Y-axis), and combinations thereof to allow for 360° rotational movement of the distal tip 206 about the wrist 208. In other applications, the pivoting motion can be limited to movement in a single plane, e.g., only pitch movement about the first axis of the wrist 208 or only yaw movement about the second axis of the wrist 208, such that the distal tip 206 moves only in a single plane.

Referring again to FIG. 2, the suction irrigator 200 may also include a plurality of drive cables (obscured in FIG. 2) that form part of a cable driven motion system configured to facilitate articulation of the distal tip 206 relative to the shaft 204. Moving (actuating) the drive cables moves the distal tip 206 between an unarticulated position and an articulated position. The distal tip 206 is depicted in FIG. 2 in the unarticulated position where a longitudinal axis A2 of the distal tip 206 is substantially aligned with a longitudinal axis A1 of the shaft 204, such that the distal tip 206 is at a substantially zero angle relative to the shaft 204.

The suction irrigator 200 may further include one or more manual actuation buttons that allow a user (e.g., a surgeon, bedside assist, etc.) to manually operate the suction irrigator 200 while on or off robot. In particular, as illustrated, the suction irrigator 200 may include a first or “irrigation” button 218a and a second or “suction” button 218b. The irrigation button 218a may be manually depressed (i.e., pushed down) to manually actuate the flow control system and thereby cause a fluid (e.g., water, a sterile saline solution, etc.) to be flowed to the distal tip 206 and discharged into a surgical site. In contrast, the suction button 218b may be manually depressed (i.e., pushed down) to manually actuate the flow control system and thereby draw a vacuum at the distal tip 206, which draws in fluids (e.g., blood, water, etc.) into the shaft 204 at the distal tip 206 from the surgical site. In some applications, the buttons 218a,b may be actuated (pressed) simultaneously during operation, or to clear clogs or prime the instrument for first use. In other embodiments, or in addition thereto, the buttons 218a,b can operate as toggle switches such that as one button 218a,b is depressed, the other is automatically disengaged (i.e., raised up).

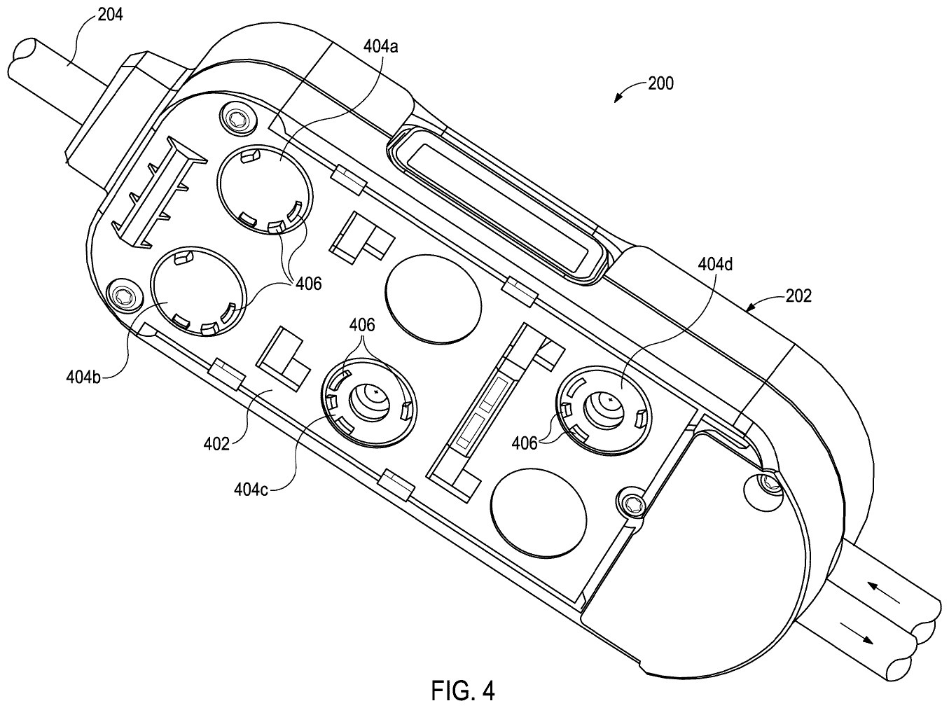

FIG. 4 is an isometric bottom view of the drive housing 202, according to one or more embodiments. As illustrated, the drive housing 202 may include a tool mounting portion 402 used to operatively couple the drive housing 202 to a tool driver of a robotic manipulator (e.g., a robot). The tool mounting portion 402 may releasably couple the drive housing 202 to the tool driver in a variety of ways, such as by clamping thereto, clipping thereto, or slidably mating therewith. In some embodiments, the tool mounting portion 402 may include an array of electrical connecting pins, which may be coupled to an electrical connection on the mounting surface of the tool driver. While the tool mounting portion 402 is described herein with reference to mechanical, electrical, and magnetic coupling elements, it should be understood that a wide variety of telemetry modalities might also be used, including infrared, inductive coupling, or the like.

The tool mounting portion 402 includes or provides an interface configured to mechanically, magnetically, and/or electrically couple the drive housing 202 to the tool driver to enable operation of the suction irrigator 200. As illustrated, the tool mounting portion 402 includes and supports a plurality of drive inputs, shown as drive inputs 404a, 404b, 404c, and 404d. Each drive input 404a-d comprises a rotatable disc configured to align with and couple to a corresponding actuator or “drive output” of a robotic tool driver, such that rotation (actuation) of a given drive output drives (rotates) a corresponding one of the drive inputs 404a-d.

Each drive input 404a-d may provide or define one or more surface features 406 configured to align with mating surface features provided on the corresponding drive output. The surface features 406 can include, for example, various protrusions and/or indentations that facilitate a mating engagement. In some embodiments, some or all of the drive inputs 404a-d may include one surface feature 406 that is positioned closer to an axis of rotation of the associated drive input 404a-d than the other surface feature(s) 406. This may help to ensure positive angular alignment of each drive input 404a-d.

In some embodiments, actuation of the first and second drive inputs 404a,b may be configured to control articulation of the wrist 208 (FIG. 2) and, therefore, the angular orientation of the distal tip 206 (FIG. 2). Actuation of the third drive input 404c may act on the flow control system housed within the drive housing 202 to facilitate irrigation; e.g., flow of a fluid through the shaft 204 to the distal tip 206, and actuation of the fourth drive input 404d may act on the flow control system to facilitate suction; e.g., generating a vacuum to draw in fluids into the shaft 204 from the surgical site.

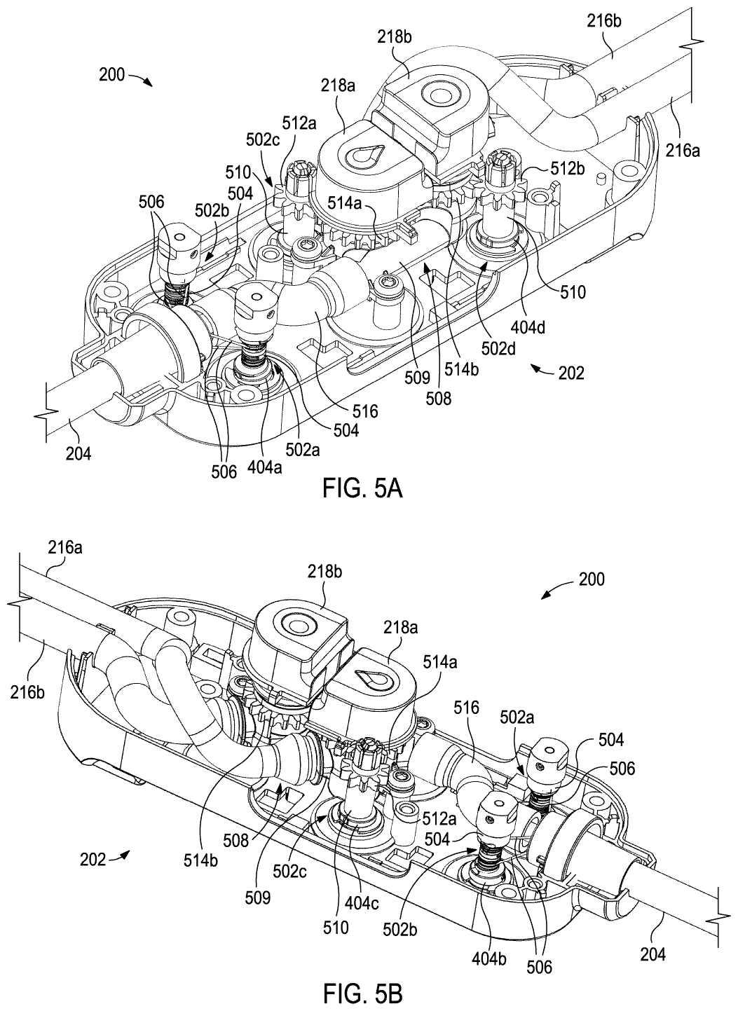

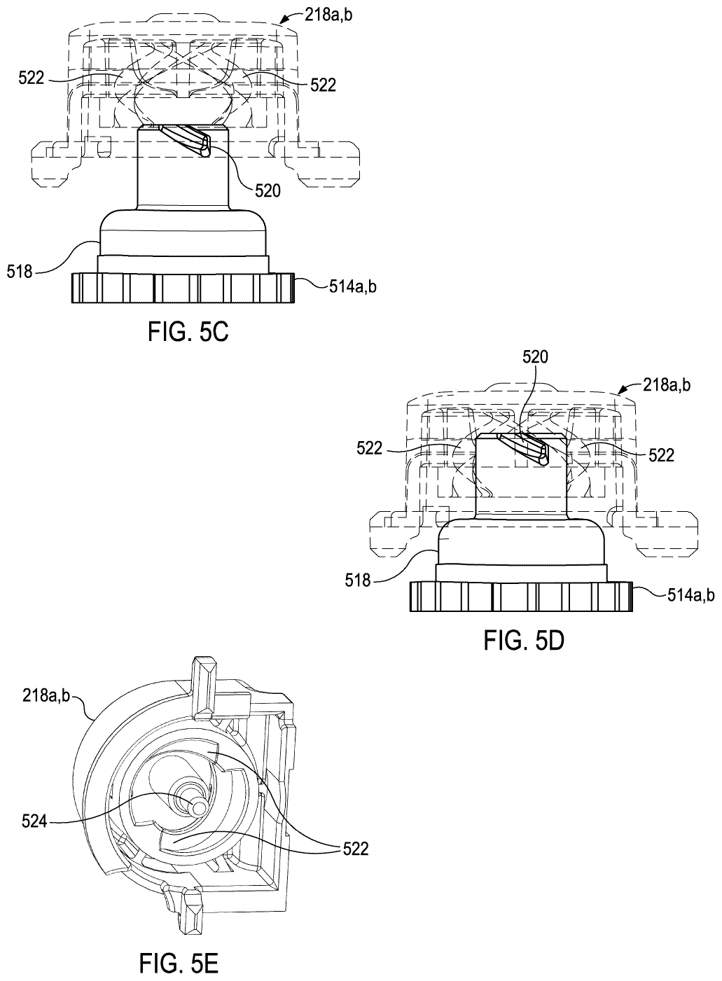

FIGS. 5A and 5B are exposed isometric views of the interior of the drive housing 202 taken from left and right vantage points, respectively, according to one or more embodiments. Several component parts that may be otherwise contained within the drive housing 202 are not shown in FIGS. 5A-5B to enable discussion of the depicted component parts.

As illustrated, the drive housing 202 houses and otherwise contains a plurality of capstan assemblies that can be actuated to operate the suction irrigator 200. In particular, the drive housing 202 contains or houses first and second capstan assemblies 502a and 502b, alternately referred to as “drive cable” capstan assemblies. The first capstan assembly 502a extends from or otherwise forms part of the first drive input 404a, and the second capstan assembly 502b extends from or otherwise forms part of the second drive input 404b. Accordingly, the first capstan assembly 502a may be actuated through operation (rotation) of the first drive input 404a, and the second capstan assembly 502b may be actuated through operation (rotation) of the second drive input 404b.

As illustrated, the first and second capstan assemblies 502a,b each include a capstan shaft 504 and one or more drive cables 506 are at least partially wrapped around each capstan shaft 504. The drive cables 506 extend into the shaft 204 and terminate at the distal tip 206 (FIG. 2). Selective actuation of the first and second capstan assemblies 502a,b applies tension (i.e., pull force) on the drive cables 506, which causes the distal tip 206 to articulate. In at least one application, the first and second capstan assemblies 502a,b may be selectively actuated to operate the drive cables 506 “antagonistically”. In such embodiments, one capstan assembly 502a,b may be actuated to pull on the corresponding drive cables 506, while the other capstan assembly 502a,b may allow the corresponding drive cables 506 to be unwound or “paid out” from the capstan shaft 504.

The drive cables 506 may comprise cables, bands, lines, cords, wires, woven wires, ropes, strings, twisted strings, elongate members, belts, shafts, flexible shafts, drive rods, or any combination thereof. The drive cables 506 can be made from a variety of materials including, but not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof. While four drive cables 506 are depicted in FIGS. 5A-5B, more or less than four may be employed.

The drive housing 202 further houses and otherwise contains a third capstan assembly 502c and a fourth capstan assembly 502d. The third capstan assembly 502c extends from or forms part of the third drive input 404c, and the fourth capstan assembly 502d extends from or forms part of the fourth drive input 404d. Accordingly, the third capstan assembly 502c may be actuated through operation (rotation) of the third drive input 404c, and the fourth capstan assembly 502d is actuated through operation (rotation) of the fourth drive input 404d.

The third and fourth capstan assemblies 502c,d are each actuatable to operate a flow control system 508 housed within the drive housing 202. As illustrated, the flow control system 508 includes a flow manifold 509 mounted within the interior of the drive housing 202. The third and fourth capstan assemblies 502c,d are each operatively coupled to the flow manifold 509 and are selectively actuatable to control the flow of fluids through the flow manifold 509 and, therefore, to and from the suction irrigator 200 . . . . As illustrated, the flow manifold 509 is in fluid communication with and otherwise fluidly coupled to the first and second conduits 216a,b. Selectively operating (actuating) the third and fourth capstan assemblies 502c,d will cause internal valves (not visible) housed within the flow manifold 509 to open or close, and thereby control the flow of fluids discharged from the shaft 204 or drawn into the shaft 204.

As illustrated, the third and fourth capstan assemblies 502c,d each include a capstan shaft 510 extending from or otherwise forming part of the third and fourth drive inputs 404c,d, respectively. A drive gear 512a and 512b may be coupled to or form part of each capstan shaft 510, such that actuation (rotation) of the corresponding drive input 404c,d will correspondingly rotate the associated drive gear 512a,b. Each drive gear 512a,b is positioned to mesh and interact with a corresponding driven gear 514a and 514b rotatably mounted to the flow manifold 509.

The first driven gear 514a may be operatively coupled to a first internal valve (not shown) included within the flow manifold 509 such that actuation (rotation) of the first driven gear 514a will correspondingly operate the first internal valve between open and closed positions. The first internal valve may be in fluid communication with the first conduit 216a such that actuation of the first internal valve controls the flow of a fluid (e.g., water, a saline solution, etc.) through the flow manifold 509. Fluids flowing through the flow manifold 509 by operation of the first internal valve are conveyed to a tube connector 516 that fluidly couples the flow manifold 509 to the shaft 204 (e.g., the inner tube of the shaft 204) for conveying the fluids to the distal tip 206 (FIG. 2). Accordingly, selectively actuating the third capstan assembly 502c will correspondingly control the irrigation function of the suction irrigator 200.

Similarly, the second driven gear 514b may be operatively coupled to a second internal valve (not shown) included within the flow manifold 509 such that actuation (rotation) of the second driven gear 514b will correspondingly operate the second internal valve between open and closed positions. The second internal valve may be in fluid communication with the second conduit 216b such that actuation of the second internal valve allows or prevents a vacuum to be generated through the flow manifold 509 and to the distal tip 206 (FIG. 2) via the tube connector 516. Accordingly, selectively actuating the fourth capstan assembly 502d will correspondingly control the suction function of the suction irrigator 200.

The irrigation and suction buttons 218a,b may also form part of the flow control system 508 and may otherwise be used to manually operate the first and second internal valves of the flow manifold 509. In conventional suction irrigators, the irrigation and vacuum control buttons are operatively and directly coupled to the third and fourth capstan assemblies 502c,d such that manually depressing the irrigation and suction buttons correspondingly acts on the third and fourth capstan assemblies 502c,d, respectively. For example, in a conventional suction irrigator, manually pressing down on the irrigation button 218a would act on and cause the first driven gear 514a to rotate, which backdrives the first drive gear 512a and further backdrives the third drive input 404c. Similarly, in a conventional suction irrigator, manually pressing down on the suction button 218b would act on and cause the second driven gear 514b to rotate, which backdrives the second drive gear 512b and further backdrives the fourth drive input 404d. When the suction irrigator is mounted to the robotic tool driver, back driving the third or fourth drive inputs 404ca,d will correspondingly backdrive the drive output of the robotic tool driver and the motor operable to drive the drive output, and back driving the motor could result in damage or complete failure of the motor. Consequently, the irrigation and suction buttons 218a,b in conventional suction irrigators may only be used when the suction irrigator is decoupled from the robotic tool driver (i.e., “off-robot) and otherwise not operably coupled to the driving motors.

According to embodiments of the present disclosure, however, features of the suction irrigator 200 and, more particularly, features of the flow control system 508 may be modified to mechanically decouple the irrigation and suction buttons 218a,b from the third and fourth capstan assemblies 502c,d. In particular, the embodiments described herein encompass several enabling and decoupling mechanisms that allow the irrigation and suction buttons 218a,b to be manually pressed while the suction irrigator 200 is mounted to the robotic tool driver, but without back driving the motors of the robotic tool driver or interfering with the controls of the robot. This may be characterized and otherwise referred to herein as “decoupling” the motion of the irrigation and suction buttons 218a,b from the robotic drive inputs. As will be appreciated, this enables “park mode” for a user (e.g., a surgeon, bedside assist, etc.), in which the surgeon is able to place or “park” the suction irrigator 200 in a known and precise orientation, clutch out of the suction irrigator 200, use both hands to drive highly articulated devices to perform another task, and simultaneously call to their bedside assist to manually actuate the suction irrigator 200 and thereby suction or irrigate without having to robotically clutch back into the suction irrigator 200.

In the embodiments shown in FIGS. 5A-5B, the flow control system 200 may further include a decoupling mechanism associated with each button 218a,b that allows the irrigation and suction buttons 218a,b to mechanically decouple from the first and second capstan assemblies 502c,d when manually pressed (actuated). This allows the irrigation and suction buttons 218a,b to be manually operated without back driving the drive inputs 404c,d against the interconnected robot motors.

FIGS. 5C-5E depict an example decoupling mechanism that may be incorporated into the flow control system 200 of FIGS. 5A-5B for either or both of the buttons 218a,b. More specifically, FIGS. 5C and 5D are side views showing the button 218a,b being manually actuated (depressed) between a first position (FIG. 5C) and a second position (FIG. 5D), and FIG. 5E is an isometric view of the interior of the button 218a,b. Moreover, the button 218a,b is shown in FIGS. 5C-5D in phantom (dashed lines) to enable viewing of internal components or structures.

As illustrated, the internal valve associated with the button 218a,b may include a valve body 518 forming part of the internal valve. In some embodiments, the valve body 518 may form an integral extension of the corresponding driven gear 514a,b and, therefore, may be configured to rotate therewith when the adjacent drive gear 512a,b (FIGS. 5A-5B) is driven by the associated capstan shaft 510 (FIGS. 5A-5B) during actuation. The button 218a,b is mounted to the valve body 518, and a helical feature 520 is defined on the valve body 518 and configured to be received within and traverse a helical profile 522 defined within the interior of the button 218a,b. Consequently, as the valve body 518 rotates, the helical feature 520 is fed into the helical profile 522, which causes the button 218a,b to move downward to actuate the internal valve. In particular, the button 218a,b may provide and otherwise define an actuation pin 524 (FIG. 5E) centrally-located within the button 218a,b and extending along a longitudinal axis of the button 218a,b. As the button 218a,b moved downward, the actuation pin 524 eventually engages and actuates the internal valve. The pin 524 is permanently fixed within the body of the valve stem. Consequently, as the button 218a,b translates up and down, the stem is correspondingly moved up and down. When the stem is up, the flow path is occluded, when the valve stem is down, the flow path is opened.

The decoupling mechanism of the flow control system 200 allows the button 218a,b to be pressed without acting on the valve body 518 and, therefore, without back driving the drive input 404c (FIGS. 5A-5B), which eliminates back driving the motor used to actuate the capstan assemblies 502c,d (FIGS. 5A-5B). In particular, when the button 218a,b is manually depressed (actuated), the button 218a,b moves downward relative to the valve body 518, and the helical feature 520 bypasses the helical profile 522, which is open to the top of the interior of the button 218a,b. Consequently, as the button 218a,b moves downward, as shown in FIG. 5D, the helical profile 522 does not engage the helical feature 520, and thereby does not act on the valve body 518, which would otherwise back drive the drive input 404c and the corresponding motor used to actuate the capstan assembly 502c,d.

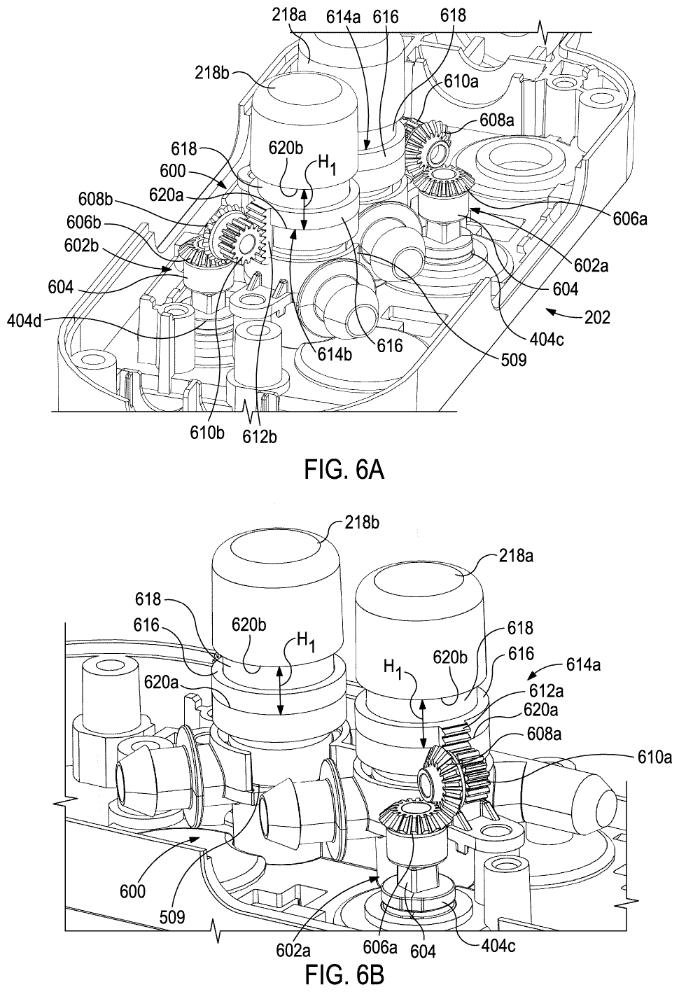

FIGS. 6A and 6B are exposed isometric views of an example flow control system 600, according to one or more embodiments of the present disclosure. The flow control system 600 may be similar in some respects to the flow control system 508 of FIGS. 5A-5B and therefore may be best understood with reference thereto. Similar to the flow control system 508, for example, the flow control system 600 may be arranged within the drive housing 202 and, therefore, may form part of the suction irrigator 200 (FIGS. 2 and 4). Moreover, the flow control system 600 includes the flow manifold 509, which houses the first and second internal valves (not visible). Furthermore, the flow control system 600 also includes the irrigation and suction buttons 218a,b used to manually operate the first and second internal valves, respectively.

It is noted, however, that several component parts that would otherwise be contained within the drive housing 202 are not shown in FIGS. 6A-6B to enable discussion of the depicted component parts. For example, the first and second conduits 216a,b (FIGS. 5A-5B) and the tube connector 516 (FIGS. 5A-5B) are omitted in FIGS. 6A-6B but would otherwise be included to facilitate the conveyance of fluids through the flow manifold 509 and to (and from) the distal tip 206 (FIG. 2).

As illustrated, the flow control system 600 includes first and second capstan assemblies 602a and 602b, where the first capstan assembly 602a extends from or forms part of the third drive input 404c, and the second capstan assembly 602b extends from or forms part of the fourth drive input 404d. Accordingly, the first capstan assembly 602a is actuated through operation (rotation) of the third drive input 404c, and the second capstan assembly 602b is actuated through operation (rotation) of the fourth drive input 404d.

The first and second capstan assemblies 602a,d are each operatively coupled to the flow manifold 509 and are selectively actuatable to control the flow of fluids therethrough. In particular, selectively operating (actuating) the first and second capstan assemblies 602a,d will cause the internal valves (not visible) within the flow manifold 509 to open or close, and thereby control the flow of fluids discharged from the shaft 204 (FIGS. 2, 4, and 5A-5B) or drawn into the shaft 204.

As illustrated, the first and second capstan assemblies 602a,b each include a capstan shaft 604 extending from or otherwise forming part of the first and second drive inputs 404c,d, respectively. A drive gear 606a and 606b may be coupled to or form part of each capstan shaft 604 such that actuation (rotation) of the corresponding drive input 404c,d will correspondingly rotate the associated drive gear 606a,b. In the illustrated embodiment, the drive gears 606a,b comprise bevel gears positioned to mesh and interact with corresponding driven gears 608a and 608b, which also comprise bevel gears. Moreover, each driven gear 608a,b may include or otherwise have formed thereon a spur gear 610a and 610b arranged to intermesh with a corresponding rack gear 612a and 612b. The rack gears 612a,b may be mounted to the irrigation and suction buttons 218a,b, respectively, such that actuation of the first and second capstan assemblies 602a,b drives the corresponding rack gear 612a,b and thereby forces the irrigation and suction buttons 218a,b downward to actuate the corresponding internal valves.

The flow control system 600 may further include a decoupling mechanism 614a and 614b associated with each button 218a,b, respectively, that allows the irrigation and suction buttons 218a,b to mechanically decouple from the first and second capstan assemblies 602a,b when manually pressed (actuated). As discussed above, this may prove advantageous in allowing the irrigation and suction buttons 218a,b to be manually operated without back driving the drive inputs 404c,d against the interconnected robot motors.

In the illustrated embodiment, each decoupling mechanism 614a,b includes a ring 616 slidably mounted to the corresponding button 218a,b within an annular recess 618 defined on the button 218a,b. The rack gear 612a,b of each capstan assembly 602a,b is fixed to the ring 616 of each decoupling mechanism 614a,b such that actuating the first and second capstan assemblies 602a,b will correspondingly act on (e.g., drive against) the ring 616 to drive the button 218a,b downward. Each annular recess 618 defines a first or “lower” shoulder 620a and a second or “upper” shoulder 620b, and each annular recess 618 exhibits a height H1 extending between the lower and upper shoulders 620a,b. The ring 616 may be configured to traverse or slide within the annular recess 618 along the height H1 and between the lower and upper shoulders 620a,b.

In example operation, actuating the first and/or second capstan assemblies 602a,b will correspondingly drive the rack gears 612a,b downward, which correspondingly drives the ring 616 downward within the annular recess 618 and against the respective lower shoulder 620a. This will simultaneously drive the corresponding buttons 218a,b downward in the same direction, which acts on and causes the internal valves (not shown) within the flow manifold 509 to actuate between closed and open positions. Deactivating actuation (ceasing operation) of the first and second capstan assembly 602a,b will allow the irrigation and suction buttons 218a,b to return to their initial position. In at least one embodiment, for example, each button 218a,b may be spring-loaded and biased to the upward position.

When it is desired to manually actuate the flow manifold 509, a user (e.g., a surgeon, bedside assist, etc.) can manually depress the appropriate irrigation or suction button 218a,b, which drives the corresponding button 218a,b downward to act on and cause the internal valves within the flow manifold 509 to actuate between closed and open positions. As the button 218a,b is manually forced downward, however, the ring 616 is able to freely travel and slide within the annular recess 618 along the height H1. The height H1 may represent “lost motion” and may be sufficient such that the ring 616 does not engage the upper shoulder 620b, and thus does not transmit a load to the corresponding the rack gear 612a,b. Consequently, this allows the buttons 218a,b to be manually depressed (actuated) without acting on the first and second capstan assembly 602a,b and, therefore, without back driving the motors used to actuate the capstan assemblies 602a,b.

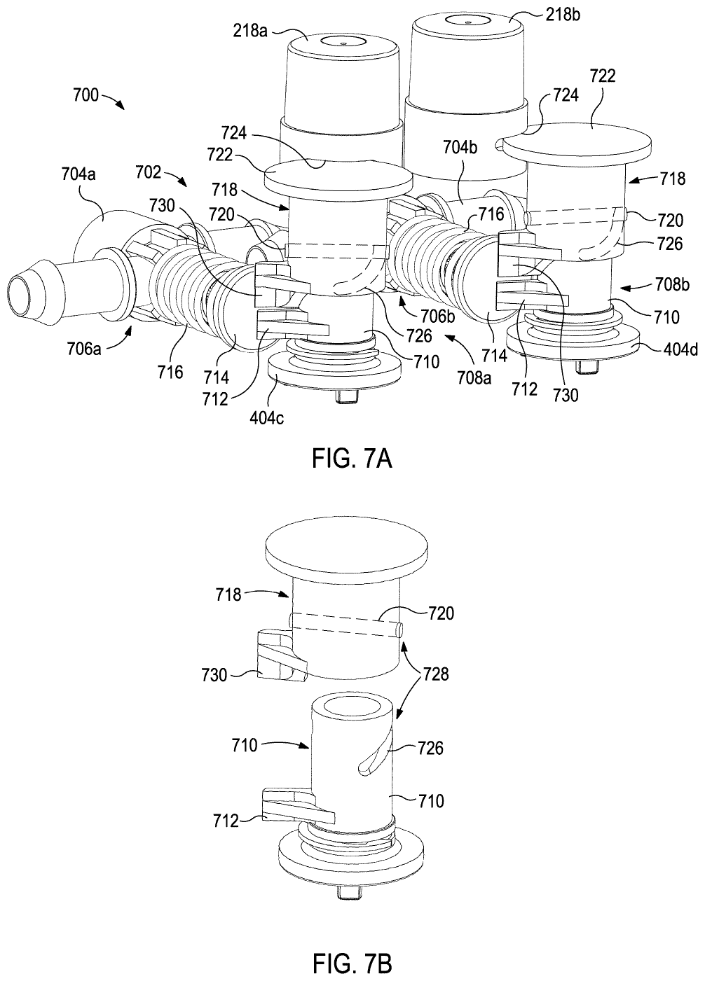

FIG. 7A is an isometric view of another example flow control system 700, according to one or more additional embodiments of the present disclosure. The flow control system 700 may be similar in some respects to the flow control system 508 of FIGS. 5A-5B and therefore may be best understood with reference thereto. Similar to the flow control system 508, for example, the flow control system 700 may be arranged within the drive housing 202 (FIGS. 2 and 4) and, therefore, may form part of the suction irrigator 200 (FIGS. 2 and 4).

As illustrated, the flow control system 700 includes a flow manifold 702, which may be similar in some respects to the flow manifold 509 (FIGS. 5A-5B). In the illustrated embodiment, the flow manifold 702 may be made of two separate parts, namely, a first or “irrigation” manifold 704a and a second or “suction” manifold 704b. In other embodiments, however, the irrigation and suction manifolds 704a,b may be integrally formed. While not shown, the first conduit 216a (FIGS. 5A-5B) may be fluidly coupled to the irrigation manifold 704a, and the second conduit 216b (FIGS. 5A-5B) may be fluidly coupled to the suction manifold 704b. A first internal valve 706a is housed within the irrigation manifold 704a, and a second internal valve 706b is housed within the suction manifold 704b. The flow control system 700 may further include the irrigation and suction buttons 218a,b used to manually operate (actuate) the first and second internal valves 706a,b, respectively.

As illustrated, the flow control system 700 includes first and second capstan assemblies 708a and 708b, which may be selectively actuated (operated) to robotically operate the first and second internal valves 706a,b. The first capstan assembly 708a extends from or forms part of the third drive input 404c, and the second capstan assembly 708b extends from or forms part of the fourth drive input 404d. Accordingly, the first capstan assembly 708a may be actuated through operation (rotation) of the third drive input 404c, and the second capstan assembly 708b is actuated through operation (rotation) of the fourth drive input 404d.

The first and second capstan assemblies 708a,d are each operatively coupled to the flow manifold 702 and are selectively operable to control the flow of fluids therethrough. More specifically, the first capstan assembly 708a may be selectively operated (actuated) to cause the first internal valve 706a to open or close, and the second capstan assembly 708b may be selectively operated (actuated) to cause the second internal valve 706b to open or close.

As illustrated, the first and second capstan assemblies 708a,b each include a capstan shaft 710 extending from or forming part of the first and second drive inputs 404c,d, respectively. A paddle or lateral arm 712 extends from each capstan shaft 710 and is engageable with a plunger 714 forming part of each internal valve 706a,b. Moving the plunger 714 in and out causes the corresponding internal valve 706a, b to transition between closed and open positions. In some embodiments, as illustrated, one or both of the plungers 714 may be spring-loaded and otherwise include a spring 716 that urges the plunger 714 to an extended position.

In example operation, as the corresponding drive input 404c,d rotates, the lateral arm 712 will correspondingly drive against the plunger 714, thereby moving the plunger 714 laterally inward to a compressed position, which actuates the respective internal valve 706a,b. As the plunger 714 moves to the compressed position, the spring 716 is compressed and builds spring force. Once the lateral force caused by the lateral arm 712 against the plunger 714 is released, the spring force of the spring 716 is able to release and thus transition the plunger 714 back to the extended position and simultaneously transition the corresponding internal valve 706a,b back to the closed position. Alternatively, the corresponding drive input 404c,d may rotate in the opposite direction, which disengages the lateral arm 712 from the plunger 714, thereby allowing the spring force of the spring 716 to naturally transition the internal valve 706a,b back to the closed position.

The first and second capstan assemblies 708a,b may each further include a cam lifter 718 operatively coupled to the capstan shaft 710. In some embodiments, as illustrated, the cam lifter 718 may be operatively coupled to the corresponding capstan shaft 710 with a camming rod or “driver” 720 (partially shown in dashed lines) extending laterally through the cam lifter 718. In particular, the camming driver 720 may be received within a cam profile 726 (shown in dashed lines) defined in the capstan shaft 710, and the camming shaft 720 may be configured to traverse the cam profile 726 as the associated button 218a,b is actuated (depressed). The cam profile 726 may comprise an angled or helical slot defined in the sidewall of the capstan shaft 710.

In some embodiments, as illustrated, the cam lifter 718 includes an enlarged head 722 operatively coupled to the associated button 218a,b. In particular, the enlarged head 722 may comprise a shoulder or lip sized to be received within a corresponding slot 724 defined in each button 218a,b. In other embodiments, however, the head 722 could alternatively be provided on the button 218a,b, and the slot 724 could instead be provided on the cam lifter 718, without departing from the scope of the disclosure. As the corresponding drive input 404c,d is actuated to operate the corresponding internal valve 706a,b, the capstan shaft 710 rotates. Since the cam driver 720 is at the end of the cam profile 726, the cam lifter 718 is caused to rotate as the drive input 404c,d rotates. Moreover, since the cam lifter 718 is not rotationally coupled to the button 218a,b, the cam lifter 718 remains essentially stationary.

FIG. 7B is an exploded view of the capstan shaft 710 and the cam lifter 718, according to one or more embodiments. As illustrated, the cam profile 726 is defined in the capstan shaft 710 and exhibits a generally angled, helical, arcuate, or curved path or trajectory. While only one cam profile 726 is shown in FIG. 7B, an opposing cam profile (not visible) is provided on the angularly opposite side of the capstan shaft 710. Consequently, when the cam lifter 718 is properly coupled to the capstan shaft 710, the cam driver 720 is extended through the cam profile 726 on both sides of the capstan shaft 710. Receiving the cam driver 720 within the cam profile 726 causes the cam driver 720 to traverse the cam profile 726 as the corresponding button 218a-b (FIG. 7A) is actuated (depressed). Forcing the cam driver 720 to traverse the cam profile 726 correspondingly rotates the cam lifter 718 and drives a paddle or lateral arm 730 against the plunger 714 (FIG. 7A) to actuate the corresponding internal valve 706a,b (FIG. 7A). During rotation, the cam lifter 718 does not act on the capstan shaft 710, but instead rotates relative thereto.

Referring again to FIG. 7A, with continued reference to FIG. 7B, the combination of the cam driver 720 and the cam profile 726 may be characterized and otherwise referred to herein as a decoupling mechanism that allows the irrigation and suction buttons 218a,b to mechanically decouple from the first and second capstan assemblies 708a,b when manually pressed (actuated). More specifically, when it is desired to manually actuate the flow manifold 702 to operate one of the internal valves 706a,b, a user (e.g., a surgeon, bedside assist, etc.) can manually depress (push down on) either of the irrigation or suction buttons 218a,b, which drives the corresponding button 218a,b downward. As the button 218a,b moves downward, the cam lifter 718 is correspondingly forced downward due to the interaction (interconnection) between the enlarged head 722 of the cam lifter 718 received within the corresponding slot 724 of the button 218a,b. Forcing the cam lifter 718 downward causes the cam driver 720 to traverse the cam profile 726, and since the capstan shaft 710 remains stationary, the cam lifter 718 will be forced to rotate relative to the capstan shaft 710 as it traverses the cam profile 726.

As illustrated, the cam lifter 718 includes a decoupled paddle or lateral arm 730 engageable against the plunger 714. As the cam lifter 718 rotates, the decoupled lateral arm 730 will correspondingly drive against the plunger 714 to actuate the corresponding internal valve 706a,b while the lateral arm 712 of the capstan shaft 710 remains stationary. Accordingly, the decoupling mechanism allows the buttons 218a,b to be manually pressed without acting on the capstan shaft 710 and thereby back driving the first and second capstan assemblies 708a,b and, therefore, without back driving the motors used to actuate the capstan assemblies 708a,b.

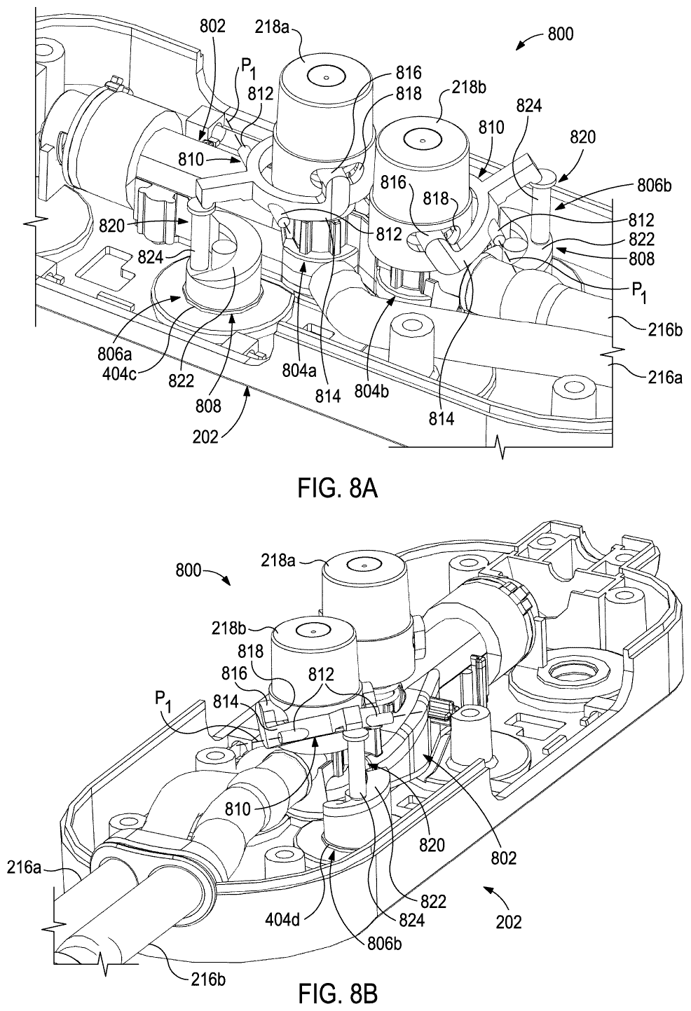

FIGS. 8A and 8B are exposed isometric views of another example flow control system 800, according to one or more additional embodiments of the present disclosure. The flow control system 800 may be similar in some respects to the flow control system 508 of FIGS. 5A-5B and therefore may be best understood with reference thereto. Similar to the flow control system 508, for example, the flow control system 800 may be arranged within the drive housing 202 and, therefore, may form part of the suction irrigator 200 (FIGS. 2 and 4). Moreover, the flow control system 800 includes a flow manifold 802, which houses first and second internal valves 804a and 804b (FIG. 8A), and the first and second conduits 216a,b are fluidly coupled to the flow manifold 802 to provide fluids or a vacuum to the flow manifold 802.

It is noted, however, that several component parts that may be otherwise contained within the drive housing 202 are not shown in FIGS. 8A-8B to enable discussion of the depicted component parts. For example, the tube connector 516 (FIGS. 5A-5B) is omitted, but would otherwise be included to facilitate the conveyance of fluids through the flow manifold 802 and to (and from) the distal tip 206 (FIG. 2).

The flow control system 800 also includes the irrigation and suction buttons 218a,b used to manually operate the first and second internal valves 804a,b, respectively, housed within the flow manifold 802. As described in more detail below, the flow control system 800 may further include a decoupling mechanism that allows the buttons 218a,b to be manually actuated (pressed down) without back driving the drive inputs 404c,d that robotically actuate the internal valves 804a,b.

As illustrated, the flow control system 800 includes first and second capstan assemblies 806a and 806b, where the first capstan assembly 806a extends from or forms part of the third drive input 404c (FIG. 8A), and the second capstan assembly 806b extends from or forms part of the fourth drive input 404d (FIG. 8B). Accordingly, the first capstan assembly 806a is actuated through operation (rotation) of the third drive input 404c, and the second capstan assembly 806b is actuated through operation (rotation) of the fourth drive input 404d.

The first and second capstan assemblies 806a,d are each operatively coupled to the flow manifold 802 and are selectively operable to control the flow of fluids therethrough by manipulating the first and second internal valves 804a,b. More specifically, the first capstan assembly 806a may be selectively operated (actuated) to cause the first internal valve 804a to open or close, and the second capstan assembly 806b may be selectively operated (actuated) to cause the second internal valve 804b to open or close.

As illustrated, the first and second capstan assemblies 806a,b each include a capstan shaft 808 extending from or forming part of the first and second drive inputs 404c,d, respectively. Each capstan shaft 808 may be operatively coupled to a corresponding one of the buttons 218a,b such that actuation (rotation) of the capstan shaft 808 causes the corresponding button 218a,b to move downward, and downward movement of the button 218a,b may actuate the associated internal valve 804a,b. In the illustrated embodiment, the capstan shafts 808 are operatively coupled to the buttons 218a,b via corresponding rocker arms 810 pivotably mounted to the drive housing 202 at one or more lateral pins 812. While not shown in FIGS. 8A-8B, a corresponding portion of the drive housing 202 may be provided to receive and pivotably mount each rocker arm 810 at the lateral pins 812. Moreover, a pivot axis P1 extends through the lateral pins 812, and as the corresponding capstan assembly 806a,b actuates, the rocker arm 810 may be configured to pivot on the lateral pins 812 about the pivot axis P1.

As illustrated, each rocker arm 810 includes one or more arms 814 that terminate in corresponding bosses 816 pivotably received within corresponding slots 818 defined in the associated buttons 218a,b. In at least one embodiment, each rocker arm 810 may include two arms 814 with corresponding bosses 816, and each arm 814 may be curved to extend about a portion of the corresponding button 218a,b to allow the bosses 816 to be received within the corresponding slots 818 provided on angularly opposite sides of the button 218a,b. As the rocker arm 810 pivots about the pivot axis P1, the bosses 816 will correspondingly pivot (rotate) within the corresponding slot 818 and thereby act on the associated button 218a,b.

The opposing end of each rocker arm 810 may include an engagement feature 820 configured to engage and traverse an arcuate cam surface 822 defined by the corresponding capstan shaft 808. The cam surface 822 comprises a ramped profile that progressively changes elevation, and the engagement feature 820 may be configured to slidably traverse the cam surface 822 as the capstan shaft 808 rotates, which causes the rocker arm 810 to pivot about the pivot axis P1. In some embodiments, the engagement feature 820 may comprise integral portion or extension of the rocker arm 810. In other embodiments, however, the engagement feature 820 may comprise a pin 824 extending between the rocker arm 810 and the capstan shaft 808. In some embodiments, the pin 824 may comprise an extension of the rocker arm 810, and may thus be formed integrally therewith. In other embodiments, however, the pin 824 may comprise a separate component part mounted within the drive housing 202 and extending between the cam surface 822 and a lateral extent of the rocker arm 810.

In example operation, as the capstan assembly 806a,b actuates (rotates), the capstan shaft 808 correspondingly rotates and causes the engagement feature 820 to slidably ride upon the cam surface 822. Slidably engaging the cam surface 822 causes the rocker arm 810 to pivot at the lateral pins 812 and about the pivot axis P1. As the rocker arm 810 pivots, the bosses 816 received within the corresponding slots 818 move vertically downward, thereby urging the associated button 218a,b in the same downward direction to actuate the corresponding internal valve 804a,b. Since the buttons 218a,b are spring-loaded, once operation (actuation) of the capstan assembly 806ab ceases, the buttons 218a,b may naturally return to the extended (upward) position under spring force.

The rocker arm 810 may form part of a decoupling mechanism that allows the irrigation and suction buttons 218a,b to decouple from the first and second capstan assemblies 806a,b when manually pressed (actuated). More specifically, when it is desired to manually actuate the flow manifold 802 or one of the internal valves 804a,b, a user (e.g., a surgeon, bedside assist, etc.) can manually depress (press down on) either of the irrigation or suction buttons 218a,b, which drives the corresponding button 218a,b downward and thereby actuates the corresponding internal valve 804a,b. As the button 218a,b moves downward, the bosses 816 received within the slots 818 are correspondingly driven downward, which causes the rocker arm 810 to pivot about the pivot axis P1 at the lateral pins 812. As the rocker arm 810 pivots about the pivot axis P1, the engagement feature 820 is disengaged from the cam surface 822, thus not acting on the capstan shaft 808. Accordingly, the decoupling mechanism allows the buttons 218a,b to be manually pressed without acting on the capstan shaft 808 or back driving the first and second capstan assemblies 806a,b, thus without back driving the motors used to actuate the capstan assemblies 806a,b.

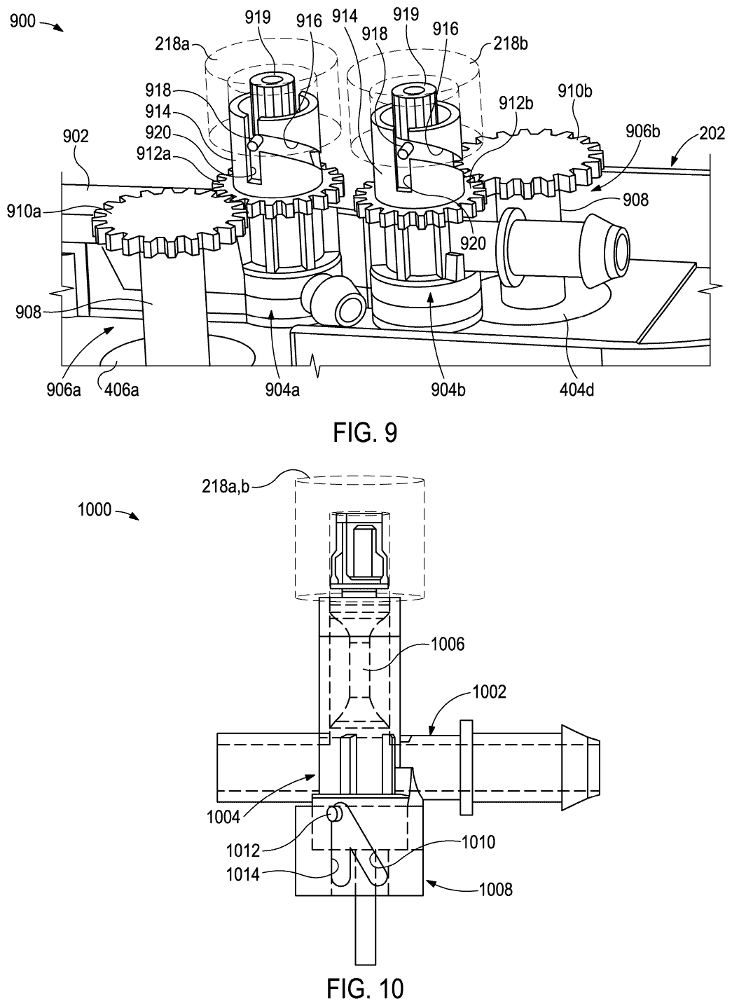

FIG. 9 is an enlarged, isometric view of another example flow control system 900, according to one or more additional embodiments of the present disclosure. The flow control system 900 may be similar in some respects to the flow control system 508 of FIGS. 5A-5B and therefore may be best understood with reference thereto. Similar to the flow control system 508, for example, the flow control system 900 may be arranged within the drive housing 202 and, therefore, may form part of the suction irrigator 200 (FIGS. 2 and 4). Moreover, the flow control system 900 includes a flow manifold 902, which houses first and second internal valves 904a and 904b. Furthermore, the flow control system 900 also includes the irrigation and suction buttons 218a,b (shown in phantom) used to manually operate the first and second internal valves 904a,b, respectively.

Several component parts that may be otherwise contained within the drive housing 202 are not shown in FIG. 9 to enable discussion of the depicted component parts. For example, the first and second conduits 216a,b (FIGS. 5A-5B) and the tube connector 516 (FIGS. 5A-5B) are omitted in FIG. 9 but would otherwise be included to facilitate the conveyance of fluids through the flow manifold 902 and to (and from) the distal tip 206 (FIG. 2).

As illustrated, the flow control system 900 includes first and second capstan assemblies 906a and 906b, where the first capstan assembly 906a extends from or forms part of the third drive input 404c, and the second capstan assembly 906b extends from or forms part of the fourth drive input 404d. Accordingly, the first capstan assembly 906a may be actuated through operation (rotation) of the third drive input 404c, and the second capstan assembly 906b is actuated through operation (rotation) of the fourth drive input 404d.

The first and second capstan assemblies 906a,d are each operatively coupled to the flow manifold 902 and are selectively operable to control the flow of fluids therethrough. In particular, selectively operating (actuating) the first and second capstan assemblies 906a,d will cause the internal valves 904a,b, respectively, within the flow manifold 902 to open or close, and thereby control the flow of fluids discharged from the shaft 204 (FIGS. 2, 4, and 5A-5B) or drawn into the shaft 204.

As illustrated, the first and second capstan assemblies 906a,b each include a capstan shaft 908 extending from or forming part of the third and fourth drive inputs 404c,d, respectively. A drive gear 910a and 910b may be coupled to or form part of each capstan shaft 908 such that actuation (rotation) of the corresponding drive input 404c,d will correspondingly rotate the associated drive gear 910a,b. Each drive gear 910a,b is positioned to mesh and interact with a corresponding driven gear 912a and 912b rotatably mounted to the flow manifold 902. The first driven gear 912a may be operatively coupled to the first internal valve 904a such that actuation (rotation) of the first driven gear 912a operates the first internal valve 904a, and the second driven gear 912b may be operatively coupled to the second internal valve 904b such that actuation (rotation) of the second driven gear 912b operates the second internal valve 904b.

The irrigation and suction buttons 218a,b may be operatively coupled to the capstan assemblies 906a,b such that actuation of the capstan assemblies 906a,b correspondingly draws the associated button 218a,b downward. More specifically, as illustrated, each button 218a,b may be mounted to a cam cylinder 914 extending from or forming part of the corresponding driven gear 912a,b. In some embodiments, as illustrated, the cam cylinder 914 may extend into the interior of the corresponding button 218a,b, or the button 218a,b may otherwise be mounted to the cam cylinder 914. Each cam cylinder 914 may provide or define an angled slot 916, and each button 218a,b may include or otherwise provide a follower pin 918 capable of being received within and traversing the angled slot 916. In some embodiments, the follower pin 918 may be provided on an interior portion of the corresponding button 218a,b and extend laterally inward to be received within the angled slot 916.

In example operation, when a given capstan assembly 906a,b is actuated (rotated), the drive gear 910a,b correspondingly rotates and drives the intermeshed driven gear 912a,b, which causes the associated cam cylinder 914 to rotate in the same direction. As the cam cylinder 914 rotates, the follower pin 918 traverses the angled slot 916, which draws the button 218a,b downward to act on and actuate the corresponding internal valve 904a,b. As illustrated, the buttons 218a,b may be in contact with a valve body 919 forming part of each corresponding internal valve 904a,b. Consequently, as the buttons 218a,b move downward, the valve body 919 is correspondingly moved in the same direction to actuate the corresponding internal valve 904a,b.

The flow control system 900 may further include a decoupling mechanism associated with each button 218a,b that allows the irrigation and suction buttons 218a,b to decouple from the first and second capstan assemblies 904a,b when manually pressed (actuated). In the illustrated embodiment, the decoupling mechanism may include a combination of the follower pin 918 and a vertical slot 920 defined in the cam cylinder 914. As illustrated, the vertical slot 920 is contiguous with the angled slot 916, but extends substantially vertical and otherwise parallel with the vertical movement of the buttons 218a,b and the valve body 919. Moreover, the button 218a,b may be mounted to the corresponding cam cylinder 914 such that the follower pin 918 aligns vertically with the vertical slot 920 when the button 218a,b is in the extended or “non-actuated” position.

When it is desired to manually actuate one of the internal valves 904a,b, a user (e.g., a surgeon, bedside assist, etc.) can manually depress the appropriate irrigation or suction button 218a,b, which drives the corresponding button 218a,b downward to act on and cause the internal valve 904a,b to actuate between closed and open positions. As the button 218a,b is manually forced downward, however, the follower pin 918 enters and traverses the vertical slot 920, and thus does not engage the angled slot 916, which would otherwise act on the corresponding capstan assembly 904a,b. Consequently, this allows the buttons 218a,b to be manually depressed (actuated) without back driving the first and second capstan assemblies 904a,b and, therefore, without back driving the motors used to actuate the capstan assemblies 904a,b.

FIG. 10 is an enlarged, isometric view of another example flow control system 1000, according to one or more additional embodiments of the present disclosure. The flow control system 1000 may be similar in some respects to the flow control system 508 of FIGS. 5A-5B and therefore may be best understood with reference thereto. Similar to the flow control system 508, for example, the flow control system 1000 may form part of the suction irrigator 200 (FIGS. 2 and 4). Moreover, the flow control system 1000 includes a flow manifold 1002 (shown in phantom), which houses at least one internal valve 1004 (only one visible). Furthermore, the flow control system 1000 also includes the irrigation and suction buttons 218a,b (only one shown in dashed lines) used to manually operate the internal valve 1004.

The flow manifold 1002 is shown in phantom to enable viewing of the internal valve 1004 and other valve components. For example, as illustrated, the internal valve 1004 may include a valve body 1006 that extends vertically within a portion of the flow manifold 1002, and the button 218a,b is operatively coupled thereto such that vertical movement of the button 218a,b correspondingly moves the valve body 1006 to actuate the internal valve 1004.

In the illustrated embodiment, the internal valve 1004 may be robotically actuated by manipulating or otherwise rotating a cam cylinder 1008 mounted to or arranged at the bottom of the flow manifold 1002. The cam cylinder 1008 may form part of or may otherwise be operatively coupled to and actuated by a capstan assembly (not shown) operable to actuate the internal valve 1004. The cam cylinder 1008 may define an angled slot 1010, and a follower pin 1012 may extend from a portion of the valve body 1006 to be received within and traverse the angled slot 1010. To robotically actuate the internal valve 1004, the capstan assembly may be actuated to rotate the cam cylinder 1008. As the cam cylinder 1008 rotates, the follower pin 1012 traverses the angled slot 1010, which draws the valve body 1006 downward to actuate the internal valve 1004. Moreover, drawing the valve body 1006 downward correspondingly pulls the interconnected button 218a,b in the same direction.

The flow control system 1000 may further include a decoupling mechanism associated with the button 218a,b that allows manual actuation of the button 218a,b to be mechanically decoupled from the capstan assembly (not shown) when manually pressed (actuated). In the illustrated embodiment, the decoupling mechanism may include a combination of the follower pin 1012 and a vertical slot 1014 defined in the cam cylinder 1008. As illustrated, the vertical slot 1014 is contiguous with the angled slot 1010, but extends substantially vertical and otherwise parallel with the vertical movement of the valve body 1006. When it is desired to manually actuate the flow manifold 1002, a user (e.g., a surgeon, bedside assist, etc.) can manually depress the appropriate irrigation or suction button 218a,b, which drives the corresponding button 218a,b and valve body 1006 downward to act on and cause the internal valve 1004 to actuate between closed and open positions. As the button 218a,b is manually forced downward, however, the follower pin 1012 traverses the vertical slot 1014, and thus does not engage the angled slot 1010, which would otherwise act on the corresponding capstan assembly. Consequently, having the follower pin 1012 traverse the vertical slot 1014 allows the button 218a,b to be manually depressed (actuated) without acting on the capstan assembly and, therefore, without back driving the motors used to actuate the capstan assembly.

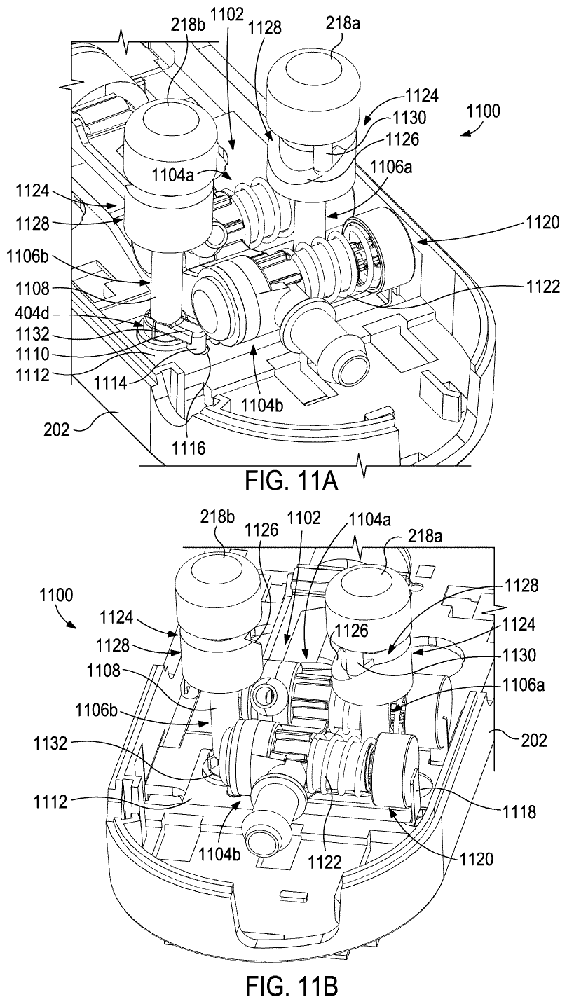

FIGS. 11A and 11B are left and right isometric views, respectively, of another example flow control system 1100, according to one or more additional embodiments of the present disclosure. The flow control system 1100 may be similar in some respects to the flow control system 508 of FIGS. 5A-5B and therefore may be best understood with reference thereto. Similar to the flow control system 508, the flow control system 1100 may be arranged within the drive housing 202 and, therefore, may form part of the suction irrigator 200 (FIGS. 2 and 4). Moreover, the flow control system 1100 includes a flow manifold 1102 that houses first and second internal valves 1104a, and the irrigation and suction buttons 218a,b may be used to manually operate (actuate) the first and second internal valves 1104a,b, respectively.

As illustrated, the flow control system 1100 includes first and second capstan assemblies 1106a and 1106b, which may be actuated (operated) to robotically operate the first and second internal valves 1104a,b, respectively. The first capstan assembly 1106a extends from or forms part of the third drive input 404c (not visible), and the second capstan assembly 1106b extends from or forms part of the fourth drive input 404d. Accordingly, the first capstan assembly 1106a may be actuated through operation (rotation) of the third drive input 404c, and the second capstan assembly 1106b is actuated through operation (rotation) of the fourth drive input 404d.

The first and second capstan assemblies 1106a,b are each operatively coupled to the flow manifold 1102 and are selectively operable to control the flow of fluids therethrough. More specifically, the first capstan assembly 1106a may be selectively operated (actuated) to cause the first internal valve 1104a to open or close, and the second capstan assembly 1106b may be selectively operated (actuated) to cause the second internal valve 1104b to open or close.

The structure and operation of the second capstan assembly 1106b will now be described, with the understanding that the description is equally applicable to the first capstan assembly 1106a. As illustrated, the second capstan assembly 1106b includes a capstan shaft 1108 extending from or forming part of the second drive input 404d. As best seen in FIG. 11A, a lateral arm 1110 extends from the capstan shaft 1108 and is engageable with a shifting substrate or “crank slider” 1112 slidably mounted to the drive housing 202. More particularly, the lateral arm 1110 may include an extension 1114 receivable within an aperture 1116 defined in the crank slider 1112. The lateral arm 1110 is operatively coupled to the crank slider 1112 such that when the second capstan assembly 1106b is actuated, the crank slider 1112 shifts laterally back and forth within the drive housing 202.

In some embodiments, the crank slider 1112 may comprise a flat sheet of rigid material, such as a metal, a plastic, a composite material, or any combination thereof. As best seen in FIG. 11B, the crank slider 1112 may be arranged beneath and otherwise extend below the flow manifold 1102, but could be arranged at other locations, without departing from the scope of the disclosure. As illustrated, the crank slider 1112 may include an engagement member 1118 engageable with a plunger 1120 forming part of the second internal valve 1104b. In some embodiments, as illustrated, the plunger 1120 may be spring-loaded and otherwise include a spring 1122 that urges the plunger 1120 to an extended position.

In example operation, as the second drive input 404d rotates, the capstan shaft 1108 will correspondingly rotate and cause the lateral arm 1110 to rotate and act on the crank slider 1112 via the interconnection (engagement) between the extension 1114 and the aperture 1116. The crank slider 1112 will shift laterally beneath the flow manifold 1102 as the lateral arm 1110 moves, and the engagement member 1118 will be driven against the face of the plunger 1120, thereby moving the plunger 1120 laterally inward to a compressed position, which actuates the second internal valve 1104b. As the plunger 1120 moves to the compressed position, the spring 1122 is compressed and builds spring force. Once the lateral force caused by the engagement member 1118 driven against the plunger 1120 is released, the spring force of the spring 1122 is able to release and drive the plunger 1120 back to the extended position while simultaneously transitioning the corresponding internal valve 1104a,b back to the closed position.

As best seen in FIG. 11A, the flow control system 1100 may further include a decoupling mechanism 1124 associated with each button 218a,b, where the decoupling mechanism 1124 is capable of decoupling manual actuation of the buttons 218a,b from the drive inputs 404c,d. More specifically, in some embodiments, the decoupling mechanism 1124 may include an angled surface 1126 defined on a portion of the capstan shaft 1108. In the illustrated embodiment, the angled surface 1126 is defined on a cylinder bushing or “camming cylinder” 1128 mounted to or otherwise forming part of the capstan shaft 1108. In other embodiments, the camming cylinder 1128 may be omitted and the angled surface 1126 may instead be provided (defined) directly on the capstan shaft 1108. The decoupling mechanism 1124 may further include a follower pin 1130 extending from the button 218a,b and engageable against the angled surface 1126 as the button 218a,b is pressed downward. In particular, pressing downward on the button 218a,b drives the follower pin 1130 against the angled surface 1126, which causes the capstan shaft 1108 to rotate. As the capstan shaft 1108 rotates, the lateral arm 1110 will be simultaneously rotated in the same angular direction and act on the engagement member 1118 to operate the internal valve 1104a,b.

Still referring to FIG. 11A, to ensure that manually pressing down on the buttons 218a,b does not drive against the corresponding drive inputs 404c,d and thereby backdrive the motors arranged to operate the drive inputs 404c,d, the decoupling mechanism 1124 may further include an arcuate channel 1132 defined on each drive input 404c,d (only the fourth drive input 404d and corresponding arcuate channel 1132 visible). The arcuate channel 1132 may have an angular magnitude (e.g., arc length) sufficient to allow the lateral arm 1110 to rotate relative to the corresponding drive input 404c,d before engaging a lateral wall or shoulder provided by the corresponding drive input 404c,d. Accordingly, the arcuate channel 1132 provides a section of lost motion where rotation of the capstan shaft 1108 does not act on the drive input 404c,d. As the lateral arm 1110 rotates from being manually actuated by pressing down on the corresponding button 218a,b, the crank slider 1112 is correspondingly shifted laterally to cause the engagement member 1118 to act on the plunger 1120 and thereby actuate the second internal valve 1104b. Accordingly, the decoupling mechanism 1124 allows the buttons 218a,b to be pressed without acting on the drive inputs 404c,d and, therefore, without back driving the motors used to actuate the capstan assemblies 1106a,b.

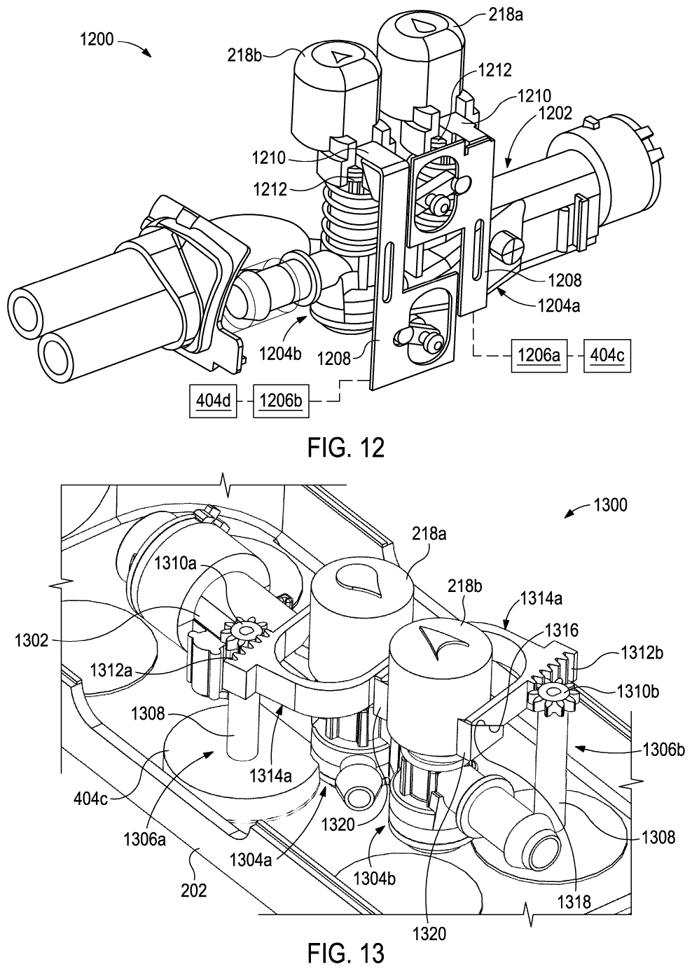

FIG. 12 is an isometric view of another example flow control system 1200, according to one or more additional embodiments of the present disclosure. The flow control system 1200 may be similar in some respects to the flow control system 508 of FIGS. 5A-5B and therefore may be best understood with reference thereto. Similar to the flow control system 508, the flow control system 1200 may form part of the suction irrigator 200 (FIGS. 2 and 4). Moreover, the flow control system 1200 includes a flow manifold 1202 that houses first and second internal valves 1204a, and the irrigation and suction buttons 218a,b are used to manually operate (actuate) the first and second internal valves 1204a,b, respectively.

The flow control system 1200 includes first and second capstan assemblies 1206a and 1206b, shown generally as boxes in FIG. 12. While shown as boxes, the capstan assemblies 1206a,b may include any of the features or designs of any of the capstan assemblies described or shown herein. As with other capstan assembly embodiments described herein, the capstan assemblies 1206a,b may be robotically actuated (operated) using the drive inputs 404c,d (shown as boxes), respectively.

The first and second capstan assemblies 1206a,b are each operatively coupled to the flow manifold 1202 and are selectively operable to control the flow of fluids therethrough. In particular, the first capstan assembly 1206a may be selectively operated (actuated) to cause the first internal valve 1204a to open or close, and the second capstan assembly 1206b may be selectively operated (actuated) to cause the second internal valve 1204b to open or close.

In the illustrated embodiment, each capstan assembly 1206a,b may include or may otherwise be operatively coupled to a corresponding shifting plate 1208 configured to shift (e.g., up and down) when the corresponding capstan assembly 1206a,b is actuated, and thereby actuate the associated internal valve 1204a,b. As illustrated, each shifting plate 1208 includes or defines a lateral arm 1210 in operable engagement (either directly or indirectly) with a valve body 1212 of the corresponding internal valve 1204a,b. In some embodiments, the lateral arm 1210 may also engage the corresponding button 218a,b, such that actuating a given capstan assembly 1206a,b not only actuates the corresponding internal valve 1204a,b via the valve body 1212, but also acts on the associated button 218a,b. In the illustrated embodiment, for example, a portion of the button 218a,b may interpose the lateral arm 1210 and the corresponding valve body 1212 of the associated internal valve 1204a,b, thus drawing the button 218a,b downward as the corresponding shifting plate 1208 is moved (actuated) downward.

In example operation, actuating the first capstan assembly 1206a causes the corresponding shifting plate 1208 to move downward and drive the lateral arm 1210 against the valve body 1212 of the first internal valve 1204a, and thereby move the valve body 1212 in the same downward direction to actuate the first internal valve 1204a. In embodiments where a portion of the irrigation button 218a interposes the lateral arm 1210 and the valve body 1212, the irrigation button 218a will simultaneously be moved in the downward direction as the lateral arm 1210 is moved downward. Similarly, actuating the second capstan assembly 1206b causes the corresponding shifting plate 1208 to move downward and drive the lateral arm 1210 against the valve body 1212 of the second internal valve 1204b, and thereby move the valve body 1212 in the same downward direction to actuate the second internal valve 1204b. In embodiments where a portion of the suction button 218b interposes the lateral arm 1210 and the valve body 1212, the suction button 218b will simultaneously be moved in the downward direction as the lateral arm 1210 is moved downward.

The flow control system 1200 may further include a decoupling mechanism that allows manual actuation of the buttons 218a,b while not back driving the capstan assemblies 1206a,b and, therefore, not back driving the motors used to drive the drive inputs 404c,d. More specifically, the decoupling mechanism for the flow control system 1200 may include the lateral arm 1210, which is unobstructed or unconstrained on its top side. Consequently, the buttons 218a,b can be manually pressed downward relative to the lateral arm 1210 and otherwise without acting on the lateral arm 1210 or the interconnected shifting plate 1208. Accordingly, the decoupling mechanism allows the buttons 218a,b to be pressed without back driving the drive inputs 404c,d and, therefore, without back driving the motors used to actuate the capstan assemblies 1206a,b.

FIG. 13 is an isometric view of another example flow control system 1300, according to one or more additional embodiments of the present disclosure. The flow control system 1300 may be similar in some respects to the flow control system 508 of FIGS. 5A-5B and therefore may be best understood with reference thereto. Similar to the flow control system 508, the flow control system 1300 may be arranged within the drive housing 202 and, therefore, may form part of the suction irrigator 200 (FIGS. 2 and 4). Moreover, the flow control system 1300 includes a flow manifold 1302 that houses first and second internal valves 1304a,b, and the irrigation and suction buttons 218a,b may be used to manually operate (actuate) the first and second internal valves 1304a,b, respectively.

The flow control system 1300 includes first and second capstan assemblies 1306a and 1306b, which may be actuated (operated) to robotically operate the first and second internal valves 1304a,b. As illustrated, each capstan assembly 1306a,b include a capstan shaft 1308 extending from or otherwise forming part of the third and fourth drive inputs 404c,d, respectively. A drive gear 1310a and 1310b may be coupled to or form part of each capstan shaft 1308, respectively, such that actuation (rotation) of the corresponding drive input 404c,d will correspondingly rotate the associated drive gear 1310a,b.

Each drive gear 1310a,b is positioned to mesh and interact with a corresponding driven gear 1312a and 1312b. As illustrated, each driven gear 1312a,b comprises a rack gear coupled to or forming part of an engagement member or “actuation wedge” 1314a and 1314b associated with each button 218a,b. In some embodiments, as illustrated, each actuation wedge 1314a,b may comprise a generally U-shaped structure that extends at least partially about the exterior of the corresponding button 218a,b. Moreover, each actuation wedge 1314a,b may provide or define one or more first angled engagement surfaces 1316 positioned to slidably engage an opposing second angled engagement surface 1318 provided on the corresponding button 218a,b. In the illustrated embodiment, the first angled engagement surfaces 1316 are provided at distal ends of opposing arms provided by the actuation wedges 1314a,b, and the opposing angled engagement surfaces 1318 are defined on wedge members 1320 (or the like) provided on angularly opposite sides of each button 218a,b.

In example operation, the capstan assemblies 1306a,b may be actuated to operate the corresponding internal valves 1304a,b. More specifically, actuating the capstan assemblies 1306a,b causes the drive gears 1310a,b to drive against the driven gears 1312a,b, which correspondingly urges the actuation wedges 1314a,b to move laterally relative to the corresponding buttons 218a,b. As the actuation wedge 1314a,b moves laterally, the opposing angled surfaces 1316, 1318 slidably engage each other, resulting in the buttons 218a,b being forced downward and simultaneously actuating the first and second internal valves 1304a,b, respectively.

The flow control system 1300 may further include a decoupling mechanism that allows manual actuation of the buttons 218a,b while not affecting the capstan assemblies 1306a,b and, therefore, not back driving the motors used to drive the drive inputs 404c,d. More specifically, the decoupling mechanism for the flow control system 1300 may include the actuation wedges 1314a,b and the opposing engagement surfaces 1316, 1318 of the actuation wedges 1314a,b and the wedge members 1320. Since the opposing engagement surfaces 1316, 1318 are not coupled to each other, the wedge members 1320 can separate from the actuation wedges 1314a,b without acting on the actuation wedges 1314a,b. Consequently, the buttons 218a,b can be manually pressed downward relative to the actuation wedges 1314a,b and otherwise without acting on the actuation wedges 1314a,b. Accordingly, the decoupling mechanism allows the buttons 218a,b to be pressed without back driving the drive inputs 404c,d and, therefore, without back driving the motors used to actuate the capstan assemblies 1306a,b.

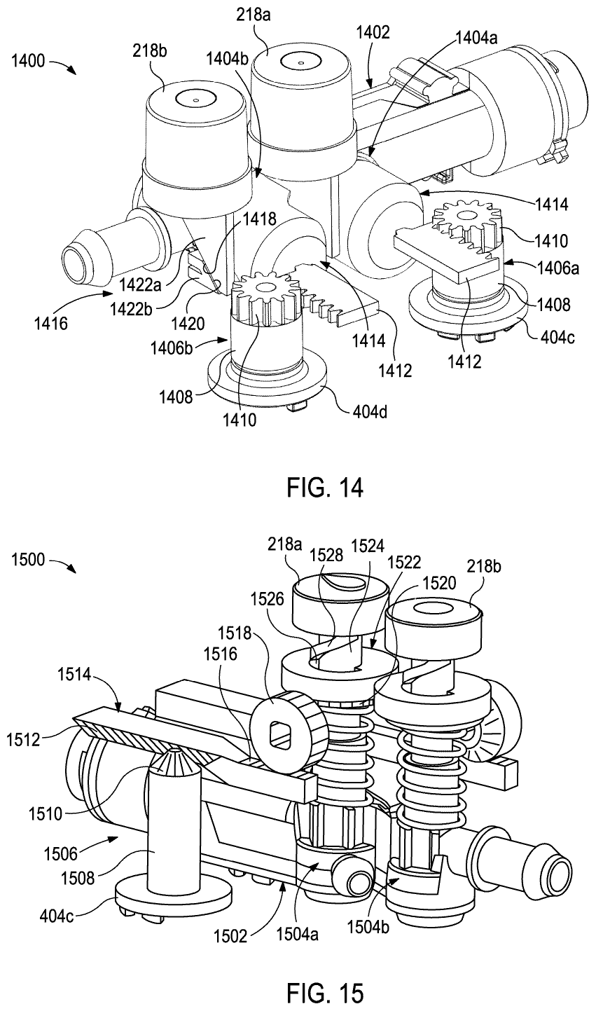

FIG. 14 is an isometric view of another example flow control system 1400, according to one or more additional embodiments of the present disclosure. The flow control system 1400 may be similar in some respects to the flow control system 508 of FIGS. 5A-5B and therefore may be best understood with reference thereto. Similar to the flow control system 508, the flow control system 1400 may form part of the suction irrigator 200 (FIGS. 2 and 4). Moreover, the flow control system 1400 includes a flow manifold 1402 that houses first and second internal valves 1404a,b, and the irrigation and suction buttons 218a,b are used to manually operate (actuate) the first and second internal valves 1404a,b, respectively.

The flow control system 1400 includes first and second capstan assemblies 1406a and 1406b, which may be actuated (operated) to robotically operate the first and second internal valves 1404a,b. As illustrated, each capstan assembly 1406a,b include a capstan shaft 1408 extending from or otherwise forming part of the third and fourth drive inputs 404c,d, respectively. A drive gear 1410 may be coupled to or form part of each capstan shaft 1408 such that actuation (rotation) of the corresponding drive input 404c,d will correspondingly rotate the associated drive gear 1410. Each drive gear 1410 is positioned to mesh and interact with a corresponding driven gear 1412. As illustrated, each driven gear 1412 comprises a rack gear operatively coupled to or otherwise engageable with a plunger 1414 forming part of the corresponding internal valve 1404a,b. In some embodiments, the plunger 1414 may be spring-loaded to urge the plunger 1414 to its extended position.

In example operation, the capstan assemblies 1406a,b may be actuated to operate the corresponding internal valves 1404a,b. More specifically, actuating the capstan assemblies 1406a,b causes the drive gears 1410 to drive against the driven gears 1412, which correspondingly urges the driven gears 1412 laterally and into engagement with the associated plunger 1414. The driven gears 1412 drive the corresponding plunger 1414 laterally inward and to a compressed position, which actuates the corresponding internal valve 1404a,b. Once the lateral force of the driven gears 1412 against the plunger 1414 is released, spring force is able to release and thus drive the plunger 1414 back to the extended position and simultaneously transition the corresponding internal valve 1404a,b back to the closed position.

The flow control system 1400 may further include a decoupling mechanism 1416 that allows manual actuation of the buttons 218a,b while not back driving the capstan assemblies 1406a,b and, therefore, not back driving the motors used to drive the drive inputs 404c,d. A description of the decoupling mechanism 1416 for the suction button 218b will now be described, with the understanding that the following description is equally applicable to the decoupling mechanism 1416 for the irrigation button 218a. As illustrated, the decoupling mechanism 1416 may include an angled engagement surface 1418 provided by the button 218b, and an opposing angled engagement surface 1420 is provided on the plunger 1414. In the illustrated embodiment, the opposing engagement surfaces 1418, 1420 are provided by opposing wedge members 1422a and 1422b provided by the button 218b and the plunger 1414.

To manually actuate the second internal valve 1404b, a user (e.g., a surgeon, bedside assist, etc.) can manually press down on the button 218b, which drives the angled engagement surface 1418 against the opposing angled engagement surface 1420, and thereby causes sliding interaction between the opposing angled engagement surfaces 1418, 1420. This sliding interaction causes the plunger 414 to move laterally inward to the compressed position, which actuates the corresponding internal valve 1404b. In this motion, the plunger 414 moves laterally away from the associated driven gear 1412, thereby not acting on the capstan assembly 1406b. Accordingly, the decoupling mechanism 1416 allows the buttons 218a,b to be pressed without back driving the drive inputs 404c,d and, therefore, without back driving the motors used to actuate the capstan assemblies 1406a,b.

FIG. 15 is an isometric view of another example flow control system 1500, according to one or more additional embodiments. The flow control system 1500 may be similar in some respects to the flow control system 508 of FIGS. 5A-5B and therefore may be best understood with reference thereto. Similar to the flow control system 508, the flow control system 1500 may form part of the suction irrigator 200 (FIGS. 2 and 4). Moreover, the flow control system 1500 includes a flow manifold 1502 that houses first and second internal valves 1504a,b, and the irrigation and suction buttons 218a,b are used to manually operate (actuate) the first and second internal valves 1504a,b, respectively.

The flow control system 1500 includes a capstan assembly 1506, which may be actuated (operated) to robotically operate the first internal valve 1504a. While not visible in FIG. 15, the flow control system 1500 also includes a second capstan assembly operable to operate the second internal valve 1504b. The following discussion related to the first capstan assembly for 1506 is equally applicable to the second capstan assembly.

As illustrated, the capstan assembly 1506 includes a capstan shaft 1508 extending from or otherwise forming part of the third drive input 404c. A drive gear 1510 may be coupled to or form part of the capstan shaft 1508 such that actuation (rotation) of the drive input 404c will correspondingly rotate the drive gear 1510. In the illustrated embodiment, the drive gear 1510 comprises a bevel gear positioned to mesh and interact with a corresponding bevel gear 1512 provided on a rack 1514 laterally (or longitudinally) movable relative to the flow manifold 1502. The rack 1514 further includes a rack gear 1516 arranged to intermesh with a corresponding pinion gear 1518. A bevel gear (occluded in FIG. 15) may form part of the pinion gear 1518 and may be arranged to intermesh with a corresponding bevel gear 1520 provided by a drive or “camming” cylinder 1522. The camming cylinder 1522 may be mounted to a valve body 1524 forming part of the first internal valve 1504a.

Actuating the capstan assembly 1506 causes the camming cylinder 1522 to rotate, which drives the valve body 1524 downward to actuate the first internal valve 1504a. More specifically, actuating the capstan assembly 1506 rotates the capstan shaft 508, which correspondingly rotates the drive gear 1510 and drives against the bevel gear 1512. Driving against the bevel gear 1512 causes the rack 1514 to move laterally, which acts on and rotates the pinion gear 1518 as engaged with the rack gear 1516. As the pinion gear 1518 rotates, the bevel gear 1520 formed on the camming cylinder 1522 is correspondingly driven, which drives the camming cylinder 1522 to rotate. As illustrated, the camming cylinder 1522 defines an internal helical profile 1526, and the valve body 1524 defines a helical feature 1528 configured to be received within and traverse the helical profile 1526. Consequently, as the camming cylinder 1522 rotates, the helical feature 1528 is fed into the helical profile 1526, which causes the valve body 1524 to move downward to actuate the first internal valve 1504. Moreover, as the valve body 1524 moves downward, the irrigation button 218a correspondingly moves in the same direction as operatively coupled to the valve body 1524.

In the embodiment of FIG. 15, the flow control system 1500 may further include a decoupling mechanism associated with each button 218a,b that allows the irrigation button 218a to mechanically decouple from the capstan assembly 1506 when manually pressed (actuated). This allows the irrigation button 218a to be manually operated without back driving the drive input 404c against the interconnected robot motor. In particular, the helical profile 1526 and the helical feature 1528 form part of the decoupling mechanism. As the valve body 1524 rotates, the helical feature 1528 is fed into the helical profile 1526, which causes the button 218a to move downward to actuate the internal valve. The decoupling mechanism allows the button 218a to be pressed without acting on the valve body 1524 and, therefore, without back driving the drive input 404c, which eliminates back driving the motor used to actuate the capstan assembly 1508. When the button 218a is manually depressed (actuated), the button 218a moves downward relative to the valve body 1524, and the helical feature 1528 bypasses the helical profile 1526, which is open to the top of the interior of the button 218a. Consequently, as the button 218a moves downward, the helical profile 1526 does not engage the helical feature 1528, and thereby does not act on the valve body 1524, which would otherwise back drive the drive input 404c and the corresponding motor used to actuate the capstan assembly 1508.

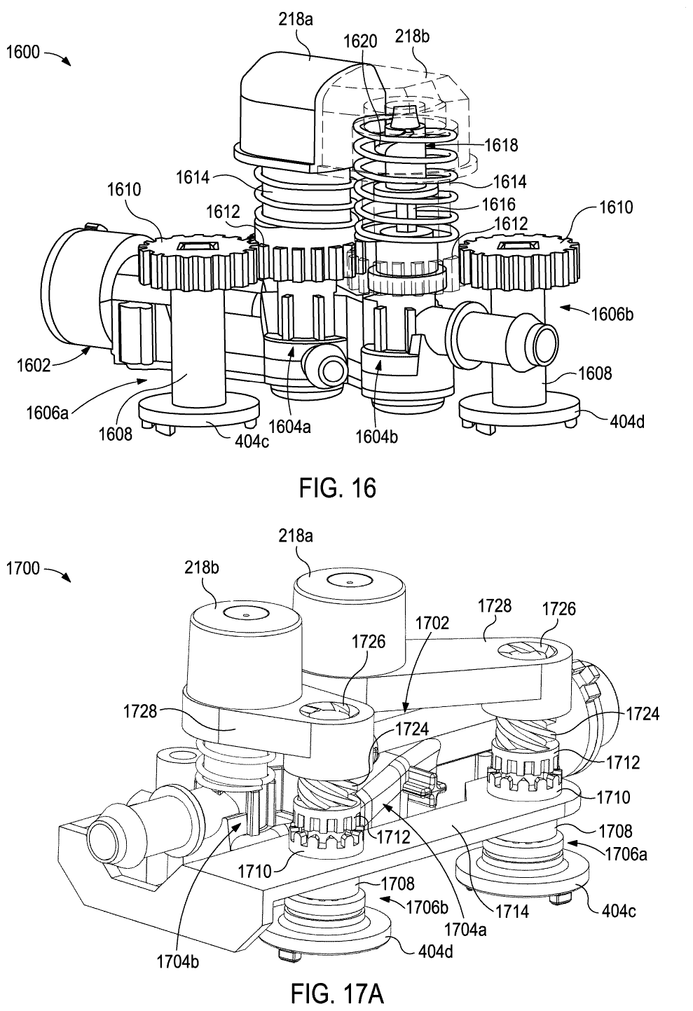

FIG. 16 is an isometric view of another example flow control system 1600, according to one or more additional embodiments of the present disclosure. The flow control system 1600 may be similar in some respects to the flow control system 508 of FIGS. 5A-5B and therefore may be best understood with reference thereto. Similar to the flow control system 508, the flow control system 1600 may form part of the suction irrigator 200 (FIGS. 2 and 4). Moreover, the flow control system 1600 includes a flow manifold 1602 that houses first and second internal valves 1604a,b, and the irrigation and suction buttons 218a,b (suction button 218b shown in phantom) are used to manually operate (actuate) the first and second internal valves 1604a,b, respectively.

The flow control system 1600 includes first and second capstan assemblies 1606a and 1606b, which may be robotically actuated (operated) to operate the first and second internal valves 1604a,b respectively. As illustrated, each capstan assembly 1606a,b includes a capstan shaft 1608 extending from or otherwise forming part of the third and fourth drive inputs 404c,d, respectively. A drive gear 1610 may be coupled to or form part of the capstan shaft 1608 such that actuation (rotation) of the drive input 404c,d will correspondingly rotate the drive gear 1610. The drive gear 1610 may be arranged to intermesh with and drive a driven gear 1612 provided on or forming part of a drive or “camming” cylinder 1614.

The camming cylinder 1614 associated with the second internal valve 1604b is shown in phantom (dashed lines) to enable viewing of internal components or structures. A valve body 1616 forming part of the second internal valve 1604b is arranged and extends within the camming cylinder 1614. The suction button 218b is mounted to the camming cylinder 1614, and a helical driver 1618 is mounted to the valve body 1616 and generally interposes the suction button 218b and the corresponding camming cylinder 1614.

As shown with reference to the suction button 218b and the second internal valve 1604b, the helical driver 1618 may be received simultaneously within portions of both the suction button 218b and the camming cylinder 1614. Moreover, the helical driver 1618 defines a helical feature 1620 configured to be received within and traverse a helical profile defined within the interior of the suction button 218b. Consequently, as the camming cylinder 1614 rotates, the helical feature 1620 is fed into the helical profile, which causes the valve body 1616 to move downward to actuate the second internal valve 1604b. Moreover, as the valve body 1616 moves downward, the interconnected suction button 218b correspondingly moves in the same direction. Operation and robotic actuation of the irrigation button 218a is undertaken similarly. Accordingly, actuating the capstan assemblies 1606a,b causes the corresponding camming cylinder 1614 to rotate, which drives the valve body 1616 downward to actuate the internal valves 1604a,b.

In the embodiment of FIG. 16, the flow control system 1600 may further include a decoupling mechanism associated with each button 218a,b that allows the button 218a,b to mechanically decouple from the corresponding capstan assembly 1606a,b when manually pressed (actuated). This allows the button 218a,b to be manually operated without back driving the corresponding drive input 404c,c against the interconnected robot motor. In particular, and with reference to the suction button 218b, the suction button 218b may define an internal helical profile (not visible) configured to receive the helical feature 1620, and the helical profile and helical feature 1620 each form part of the decoupling mechanism. As the valve body 1616 rotates, the helical driver 1618 correspondingly rotates and the helical feature 1620 is fed into the helical profile. This causes the button 218b to move downward to actuate the internal valve 1604b. The decoupling mechanism allows the button 218b to be pressed without acting on the valve body 1616 or the helical driver 1618 and, therefore, without back driving the drive input 404d, which eliminates back driving the motor used to actuate the capstan assembly 1606b. When the button 218b is manually depressed (actuated), the button 218b moves downward relative to the valve body 1616 and the helical driver 1618, and the helical feature 1620 bypasses the helical profile, which is open to the top of the interior of the button 218b. Consequently, as the button 218b moves downward, the helical profile does not engage the helical feature 1620, and thereby does not act on the valve body 1616, which would otherwise back drive the drive input 404d and the corresponding motor used to actuate the capstan assembly 1606b.

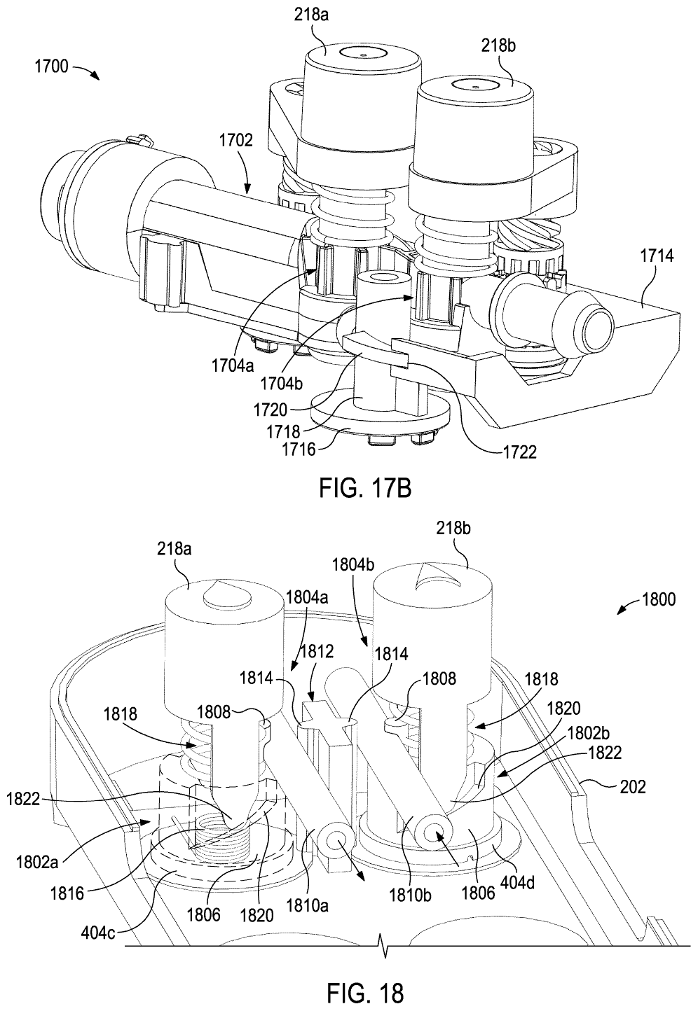

FIGS. 17A and 17B are left and right isometric views, respectively, of another example flow control system 1700, according to one or more additional embodiments of the present disclosure. The flow control system 1700 may be similar in some respects to the flow control system 508 of FIGS. 5A-5B and therefore may be best understood with reference thereto. Similar to the flow control system 508, the flow control system 1700 may form part of the suction irrigator 200 (FIGS. 2 and 4). Moreover, the flow control system 1700 includes a flow manifold 1702 that houses first and second internal valves 1704a,b, and the irrigation and suction buttons 218a,b are used to manually operate (actuate) the first and second internal valves 1704a,b, respectively.

As best seen in FIG. 17A, the flow control system 1700 includes first and second capstan assemblies 1706a and 1706b, which may be robotically actuated (operated) to operate the first and second internal valves 1704a,b. As illustrated, each capstan assembly 1706a,b include a capstan shaft 1708 extending from or otherwise forming part of the third and fourth drive inputs 404c,d. A drive gear 1710 may be coupled to or form part of each capstan shaft 1708 such that actuation (rotation) of the corresponding drive input 404c,d will correspondingly rotate the associated drive gear 1710.

Still referring to FIG. 17A, each drive gear 1710 is positioned to mesh and interact with a corresponding driven gear 1712. In the illustrated embodiment, the drive and driven gears 1710, 1712 are provided as matable castellations or castellated features. Moreover, the interface between the drive and driven gears 1710, 1712 may be selectively engaged or disengaged (separated) via an actuatable clutch plate 1714 upon which the drive gears 1710 are rotatably mounted. When the clutch plate 1714 is actuated in a first direction (e.g., upward), the drive gear 1710 mates and intermeshes with the driven gear 1712 such that rotation of the drive gear 1710 will correspondingly rotate the driven gear 1712. In contrast, when the clutch plate 1714 is actuated in a second direction (e.g., downward), the drive gear 1710 disengages from the driven gear 1712 such that rotating the drive gear 1710 will not rotate the driven gear 1712.

Referring to FIG. 17B, the flow control system 1700 may further include a clutch drive input 1716 operatively coupled to the clutch plate 1714 such that actuation of the clutch drive input 1716 causes the clutch plate 1714 to move in the first or second directions. The clutch drive input 1716 may be similar to the third and fourth drive inputs 404c,d (FIG. 17A), and may thus be actuated (operated) by a drive output (not shown) forming part of a robotic motor forming part of a robotic manipulator. As illustrated, a drive shaft 1718 extends from the clutch drive input 1716 and is operatively coupled to the clutch plate 1714 such that actuation of the clutch drive input 1716 correspondingly raises or lowers the clutch plate 1714. More specifically, a helical profile 1720 may be defined on or otherwise provided by the drive shaft 1718, and the helical profile 1720 may slidably mate with a profile slot 1722 defined in a portion of the clutch plate 1714. In example operation, as the clutch drive input 1716 is actuated, the drive shaft 1718 correspondingly rotates and the helical profile 1714 slides within (traverses) the profile slot 1722, which causes the clutch plate 1714 to raise or lower, depending on the rotational direction of the drive shaft 1718.

Referring again to FIG. 17A, with continued reference to FIG. 17B, example operation of the flow control system 1700 is now provided. To actuate the internal valves 1704a,b, the clutch drive input 1716 may first be actuated to raise the clutch plate 1714 and thereby engage the drive gears 1710 with the driven gears 1712. As illustrated, each driven gear 1712 is coupled to or otherwise forms part of a helical gear 1724 received within a helical thread profile 1726 defined in an arm 1728 extending laterally from each button 218a,b. The helical gear 1724 is secured in the vertical direction, but is allowed to rotate relative to the arm 1728. While not shown, in some embodiments, for example, the helical gear 1724 may be secured in place by engagement with the surrounding drive housing. Alternatively, the helical gear 1724 could be held in place via the capstan assembly 1706a,b via suitably positioned tabs. Consequently, rotating the helical gear 1724 does not change its vertical position, but instead acts on the arm 1728 through which the helical gear 1724 is threaded.

Accordingly, when the drive and driven gears 1710, 1712 are mated, actuating the capstan assemblies 1706a,b causes the drive gears 1710 to drive against the driven gears 1712 and thereby rotate the helical gears 1724. Rotating the helical gears 1724 advances the helical gears 1724 into the corresponding helical thread profile 1726, which draws the associated button 218a,b downward. The buttons 218a,b may be engageable with the internal valves 1704a,b, respectively, such that as the buttons 218a,b are drawn downward, the corresponding internal valve 1704a,b is actuated.

The flow control system 1700 may further include a decoupling mechanism that allows manual actuation of the buttons 218a,b while not back driving the capstan assemblies 1706a,b, and therefore not back driving the motors used to drive the drive inputs 404c,d. The decoupling mechanism for the flow control system 1700 includes the clutch plate 1714 and the ability to move the drive gears 1710 into and out of engagement with the driven gear 1712. More particularly, when it is desired to manually actuate the internal valves 1704a,b, the clutch drive input 1716 may be actuated to lower the clutch plate 1714, and thereby disengage the drive gear 1710 from the driven gear 1712. With the drive gears 1710 disengaged from the driven gears 1712, a user may manually press down on either of the buttons 218a, b to manually actuate the corresponding internal valves 1704a,b. As the button 218a,b is moved downward, the lateral arm 1728 is correspondingly moved downward, which causes the helical gear 1724 to rotate and advance into the corresponding helical thread profile 1726. Rotating the helical gear 1724 causes the associated driven gear 1712 to rotate in the same direction. However, since the driven gears 1712 are not engaged with the drive gears 1710, rotating the helical gears 1724 will not backdrive the corresponding capstan assembly 1706a,b. Accordingly, the decoupling mechanism allows the buttons 218a,b to be pressed without back driving the drive inputs 404c,d and, therefore, without back driving the motors used to actuate the capstan assemblies 1706a,b.

FIG. 18 is an exposed, isometric view of yet another flow control system 1800, according to one or more additional embodiments of the present disclosure. The flow control system 1800 may be similar in some respects to one or more of the flow control systems described herein. For example, as illustrated, the flow control system 1800 may be arranged within the drive housing 202 and, therefore, may form part of the suction irrigator 200 (FIGS. 2 and 4). Moreover, the flow control system 1800 includes the irrigation and suction buttons 218a,b, which can be used to manually operate (actuate) the functionality of the suction irrigator 200.

As illustrated, the flow control system 1800 includes first and second capstan assemblies 1802a and 1802b, which may be actuated (operated) to robotically operate first and second pinch valves 1804a,b, respectively. As illustrated, each capstan assembly 1802a,b includes a capstan shaft 1806 extending from or otherwise forming part of the third and fourth drive inputs 404c,d, respectively. Accordingly, actuating (rotating) the drive inputs 404c,d will correspondingly rotate the capstan shafts 1806.

A pinch member 1808 is mounted to or otherwise forms an integral part of each capstan shaft 1806, and is rotatable therewith when actuated. The pinch members 1808 form part of the pinch valves 1804a,b. More specifically, as illustrated, the flow control system 1800 includes first and second flow tubes 1810a and 1810b, where the first flow tube 1810a is associated with the irrigation button 218a and the first capstan assembly 1802a, and the second flow tube 1810b is associated with the suction button 218b and the second capstan assembly 1802b. A pinch rib 1812 may form a common part of each of the pinch valves 1804a,b, and may provide and otherwise define opposing pinch protrusions 1814 extending laterally outward from the main body of the pinch rib 1812.

In example operation, the capstan assemblies 1802a,b may be actuated to actuate (operate) the pinch valves 1804a,b, respectively. More specifically, when the capstan assembly 1802a,b is actuated, the capstan shaft 1806 is rotated, which correspondingly rotates the pinch member 1808 into lateral engagement with the corresponding flow tube 1810a,b. This will effectively pinch the corresponding flow tube 1810a,b between the pinch member 1808 and the opposing pinch protrusion 1814 provided on the pinch rib 1812, and will stop fluid flow through the flow tube 1810a,b. Reversing the rotational direction of the capstan shaft 1806 will move the pinch member 1808 out of engagement with the flow tube 1810a,b, thereby restoring fluid flow capability through the corresponding flow tube 1810a,b.

The capstan shaft 1806 of the first capstan assembly 1802a is shown in dashed lines to enable viewing of internal component. As illustrated, in some embodiments, a torsion spring 1816 may be arranged within the capstan shaft 1806. Torsion spring helps maintain the pinch valves 1804a,b in a default closed state.

The flow control system 1800 may further include a decoupling mechanism 1818 that allows manual actuation of the buttons 218a,b while not back driving the capstan assemblies 1802a,b, and therefore not back driving the motors used to drive the drive inputs 404c,d. In the illustrated embodiment, the decoupling mechanism 1818 includes an angled or helical profile 1820 defined in the capstan shaft 1806, and a follower pin 1822 operatively coupled to or forming an integral part of each button 218a,b and configured to slidably engage the helical profile 1820. When it is desired to manually actuate the pinch valves 1804a,b, a user (e.g., a surgeon, bedside assist, etc.) may press down on the button 218a,b, which drives the follower pin 1822 to slidingly engage the helical profile 1820 and thereby cause the corresponding capstan shaft 1806 to rotate. Rotating the capstan shaft 1806 moves the corresponding pinch member 1808 into lateral engagement with the corresponding flow tube 1810a,b and effectively pinches the flow tube 1810a,b between the pinch member 1808 and the opposing pinch protrusion 1814 provided on the pinch rib 1812. The drive inputs 404c,d are separate bodies from capstan assemblies 1802a,b. Thus, pressing down on the button 218a,b will result in a rotation of the capstan assemblies 1802a,b, but not the drive inputs 404c,d. There is free relative movement between those bodies, and a rotational hardstop that prevents the capstan assemblies 1802a,b from rotating clockwise on the drive inputs 404c,d. Accordingly, the decoupling mechanism 1818 allows the buttons 218a,b to be pressed without back driving the drive inputs 404c,d and, therefore, without back driving the motors used to actuate the capstan assemblies 1802a,b.

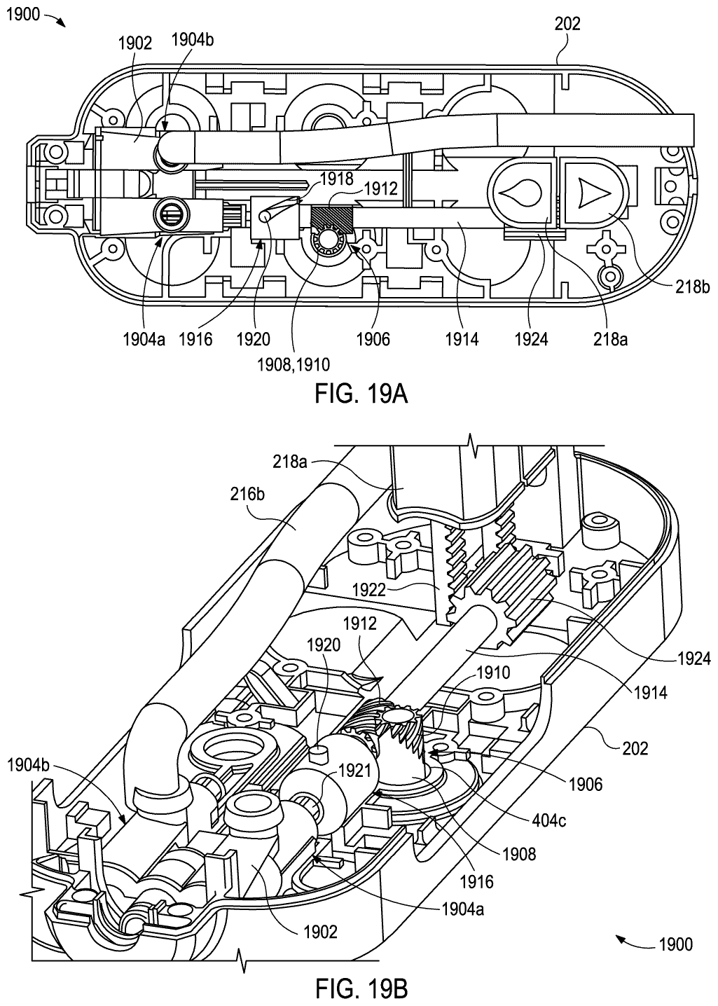

FIGS. 19A and 19B are top and isometric views, respectively, of another example flow control system 1900, according to one or more additional embodiments of the present disclosure. The flow control system 1900 may be similar in some respects to the flow control system 508 of FIGS. 5A-5B and therefore may be best understood with reference thereto. Similar to the flow control system 508, the flow control system 1900 may be arranged within the drive housing 202 and may form part of the suction irrigator 200 (FIGS. 2 and 4). Moreover, the flow control system 1900 includes a flow manifold 1902 that houses first and second internal valves 1904a,b, and the irrigation and suction buttons 218a,b are used to manually operate (actuate) the first and second internal valves 1904a,b, respectively.

Several component parts that may be otherwise contained within the drive housing 202, however, are not shown in FIGS. 19A-19B to enable discussion of the depicted component parts. For example, while the second conduit 216b (shown in dashed lines) is depicted, the first conduit 216a (FIGS. 5A-5B) and the tube connector 516 (FIGS. 5A-5B) are omitted in FIGS. 19A-19B, but would otherwise be included to facilitate the conveyance of fluids through the flow manifold 1902 and to (and from) the distal tip 206 (FIG. 2).

The flow control system 1900 includes a capstan assembly 1906 that may be actuated (operated) to robotically operate the first internal valve 1904a. While not shown in FIGS. 19A-19B, a second capstan assembly may also be included in the flow control system 1900 to robotically operate the second internal valve 1904b. The following discussion related to the first internal valve 1904a may be equally applicable to the non-depicted second capstan assembly to operate the second internal valve 1904b.

As illustrated, the capstan assembly 1906 includes a capstan shaft 1908 extending from or otherwise forming part of the third drive input 404c (see FIG. 19B, mostly occluded). A drive gear 1910 may be coupled to or form part of the capstan shaft 1908 such that actuation (rotation) of the drive input 404c will correspondingly rotate the drive gear 1910. The drive gear 1910 may be arranged to intermesh with and drive a driven gear 1912 provided on a drive shaft 1914 arranged within the drive housing 202. In some embodiments, the driven gear 1912 comprises an outer sleeve mounted to the drive shaft 1914 and configured to translate axially (e.g. left and right in FIG. 19A), but fixed rotationally. In the illustrated embodiment, the drive and driven gears 1910, 1912 comprise corresponding helical gears. In other embodiments, however, the drive and driven gears 1910, 1912 may comprise other types of geared interfaces, without departing from the scope of the disclosure.

The flow control system 1900 may further include a driven cam member 1916 mounted to the distal end of the drive shaft 1914. As best seen in FIG. 19A, a helical slot 1918 may be defined in the driven cam member 1916, and the drive shaft 1914 provides a follower pin 1920 capable of traversing the helical slot 1918 as the drive shaft 1914 rotates. In example operation to robotically actuate the first internal valve 1904a, the capstan assembly 1906 is actuated to rotate the capstan shaft 1908, which correspondingly rotates the drive gear 1910 and drives against the driven gear 1912. Driving the driven gear 1912 causes the driven gear 1912 to axially translate along the drive shaft 1914 and axially engage the driven cam member 1916. The driven cam member 1916 is thereby driven distally relative to the drive shaft 1914 (which does not move) to engage and push against a valve body 1921 of the internal valve 1904a, which operates the internal valve 1904a between closed and open positions.

The flow control system 1900 also includes a means of manually actuating the first internal valve 1904a. More specifically, the flow control system 1900 includes a rack gear 1922 (see FIG. 19B) extending from the irrigation button 218a and engageable with a pinion gear 1924 coupled to or otherwise forming part of the drive shaft 1914. To manually actuate the first internal valve 1904a, a user (e.g., a surgeon, bedside assist, etc.) may manually press down on the irrigation button 218a, which correspondingly drives the rack gear 1922 downward. Driving the rack gear 1922 downward drives against the pinion gear 1924, which causes the drive shaft 1914 to rotate. As the drive shaft 1914 rotates, the follower pin 1920 traverses the helical slot 1918 of the driven cam member 1916, which causes the driven cam member 1916 to move distally and into engagement with the internal valve 1904a to operate the internal valve 1904a between closed and open positions.]

Accordingly, when a user pushes down on the irrigation button 218a, the rack gear 1922 engages the pinion gear 1924, which causes the drive shaft 1914 to rotate. Since the driven gear 1912 comprises a sleeve mounted to the drive shaft 1914 such that it is rotationally fixed but able to axially translate, the irrigation button 218a cab be pressed without back driving the drive input 404c and, therefore, without back driving the motor used to actuate the capstan assemblies 1906a,b.

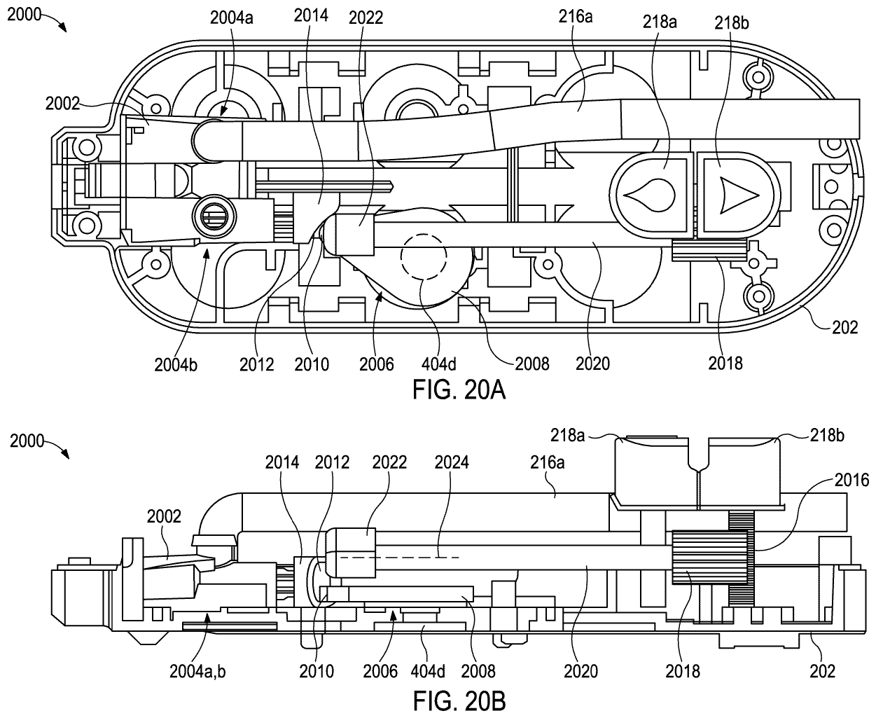

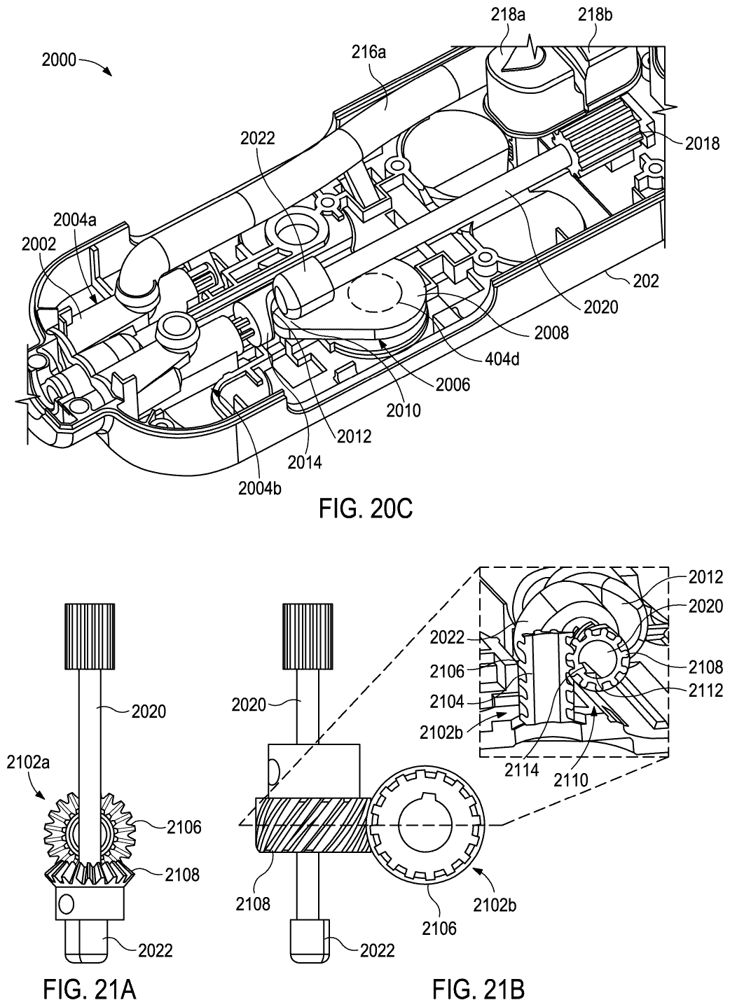

FIGS. 20A-20C are exposed top, side, and isometric views, respectively, of another example flow control system 2000, according to one or more additional embodiments of the present disclosure. The flow control system 2000 may be similar in some respects to the flow control system 508 of FIGS. 5A-5B and therefore may be best understood with reference thereto. Similar to the flow control system 508, the flow control system 2000 may be arranged within the drive housing 202 and may form part of the suction irrigator 200 (FIGS. 2 and 4). Moreover, the flow control system 2000 includes a flow manifold 2002 that houses first and second internal valves 2004a,b, and the irrigation and suction buttons 218a,b are used to manually operate (actuate) the first and second internal valves 2004a,b, respectively.

Several component parts that may be otherwise contained within the drive housing 202, however, are not shown in FIGS. 20A-20C to enable discussion of the depicted component parts. For example, while the first conduit 216a (shown in dashed lines) is depicted, the second conduit 216b (FIGS. 5A-5B) and the tube connector 516 (FIGS. 5A-5B) are omitted in FIGS. 20A-20C, but would otherwise be included to facilitate the conveyance of fluids through the flow manifold 2002 and to (and from) the distal tip 206 (FIG. 2).

The flow control system 2000 includes a capstan assembly 2006 that may be robotically actuated (operated) to operate the second internal valve 2004b. While not shown in FIGS. 20A-20C, a second capstan assembly may also be included in the flow control system 2000 to robotically operate the first internal valve 2004a. The following discussion related to the second internal valve 2004b may be equally applicable to the non-depicted second capstan assembly to operate the first internal valve 2004a.

As illustrated, the capstan assembly 2006 includes a cam driver 2008 extending from or otherwise forming part of the fourth drive input 404d (shown in dashed lines in FIGS. 20A and 20B) such that actuation (rotation) of the fourth drive input 404d correspondingly rotates the cam driver 2008. The cam driver 2008 may exhibit an oblong or irregular shape that provides or otherwise defines a camming lobe 2010 engageable with an arcuate camming surface 2012 defined on a plunger 2014 that forms part of the second internal valve 2004b. In example operation to robotically actuate the second internal valve 2004b, the capstan assembly 2006 is actuated to rotate the cam driver 2008. As the cam driver 2008 rotates, the camming lobe 2010 is rotated into engagement with the camming surface 2012 of the plunger 2014, which urges the plunger 2014 to move distally to engage and actuate the second internal valve 2004b.

The flow control system 2000 also includes a means of manually actuating the second internal valve 2004b without back driving the capstan assembly 2006. More specifically, the flow control system 2000 includes a rack gear 2016 (FIG. 20B) extending from the suction button 218b and engageable with a pinion gear 2018 coupled to or otherwise forming part of a drive shaft 2020 extending longitudinally within the drive housing 202. A driven cam member 2022 may be provided at a distal end of the drive shaft 2020 and may be engageable with the camming surface 2012 of the plunger 2014 when the suction button 218b is manually depressed.

In some embodiments, the driven cam member 2022 may be coupled to the drive shaft 2020 such that rotating the drive shaft 2020 will urge the driven cam member 2022 into engagement with the camming surface 2012. In such embodiments, the driven cam member 2022 may be cylindrical and have a substantially circular cross-section, but may be coupled to the drive shaft 2020 such that it is offset from a longitudinal axis 2024 (FIG. 20B) of the drive shaft 2020. As a result, rotating the drive shaft 2020 will drive the offset portion of the driven cam member 2022 into engagement with the camming surface 2012 and thereby urge the plunger 2014 to move. In other embodiments, however, the driven cam member 2022 may exhibit an oblong or ovoid (e.g., egg-shaped) cross-sectional shape, where a portion of the driven cam member 2022 is offset from the longitudinal axis 2024 and engageable with the camming surface 2012 when the drive shaft 2020 is rotated.

To manually actuate the second internal valve 2004b, a user (e.g., a surgeon, bedside assist, etc.) manually depresses (i.e., pushed shown) the suction button 218b, which correspondingly drives the rack gear 2016 downward. Driving the rack gear 2016 downward correspondingly drives against the pinion gear 2018, which causes the drive shaft 2020 to rotate. As the drive shaft 2020 rotates, the driven cam member 2022 is urged into engagement with the camming surface 2012 of the plunger 2014, which causes the plunger 2014 to move distally and actuate the second internal valve 2004b. Since the drive shaft 2020 and interconnected driven cam member 2022 are entirely separate from the capstan assembly 2006, the suction button 218b can be manually actuated (pressed) without back driving the drive input 404d and, therefore, without back driving the motor used to actuate the capstan assembly 2006.

Referring now to FIGS. 21A and 21B, with continued reference to FIGS. 20A-20C, illustrated are alternative capstan assemblies 2102a and 2102b, respectively, which may be used in conjunction with the flow control system 2000. In each embodiment, the capstan assembly 2102a,b may include a capstan shaft 2104 (shown in inset graphic of FIG. 21B) that may extend from or form part of one of the third or fourth drive inputs 404c,d (FIGS. 5A-5B). Moreover, the capstan shaft 2104 may have a drive gear 2106 coupled thereto or forming part thereof, and the drive gear 2106 may be arranged to intermesh with a corresponding driven gear 2108 mounted to the drive shaft 2020.

In FIG. 21A, the drive and driven gears 2106, 2108 comprise matable bevel gears. In FIG. 21B, the drive and driven gears 2106, 2108 comprise matable helical gears. In example operation, the drive gear 2106 drives the driven gear 2108, which causes the drive shaft 2020 to rotate, thereby driving the driven cam member 2022 into engagement with the camming surface 2012 (FIGS. 20A-20C) of the plunger 2014 (FIGS. 20A-20C), and thereby causes the plunger 2014 to move distally and actuate the second internal valve 2004b (FIGS. 20A-20C), as generally described above.

In some embodiments, as shown in the inset graphic of FIG. 21B, the second capstan assembly 2102b may further include a decoupling mechanism 2110 that allows the suction button 218b to be manually depressed without back driving the second capstan assembly 2102b. While described with reference to the second capstan assembly 2102b, the decoupling mechanism 2110 is equally applicable to the first capstan assembly 2102a. In the illustrated embodiment, the decoupling mechanism 2110 includes an arcuate channel 2112 defined in the drive shaft 2020, and the driven gear 2108 includes a projection 2014 receivable within the arcuate cutout 2112. When the second capstan assembly 2102b is robotically actuated, the drive gear 2106 drives against the driven gear 2108, and the projection 2014 engages a lateral wall or shoulder of the arcuate channel 2112, which transfers the rotational motion of the driven gear 2108 to the drive shaft 2020. As the drive shaft 2020 rotates, the driven cam member 2022 is urged into engagement with the camming surface 2012, which causes the plunger 2014 to move distally and actuate the second internal valve 2004b.

In contrast, when the drive shaft 2020 is manually rotated by manually actuating (depressing) the suction button 218b (FIGS. 20A-20C), as generally described above, the projection 2014 may traverse the arcuate channel 2112 and thereby allow the drive shaft 2020 to rotate relative to the driven gear 2108. The arcuate channel 2112 may exhibit an angular magnitude (e.g., arc length) sufficient to allow to the drive shaft 2020 to rotate relative to the driven gear 2108 without the projection 2014 engaging a lateral wall or shoulder of the arcuate channel 2112 and, therefore, without acting on the capstan assembly 2102b. Accordingly, the arcuate channel 2112 provides a section of lost motion where rotation of the drive shaft 2020 does not act on the capstan shaft 2104. Accordingly, the decoupling mechanism 2110 allows the button 218b to be pressed without back driving the drive input 404d (FIGS. 5A-5B) and, therefore, without back driving the motors used to actuate the capstan assembly 2102b.

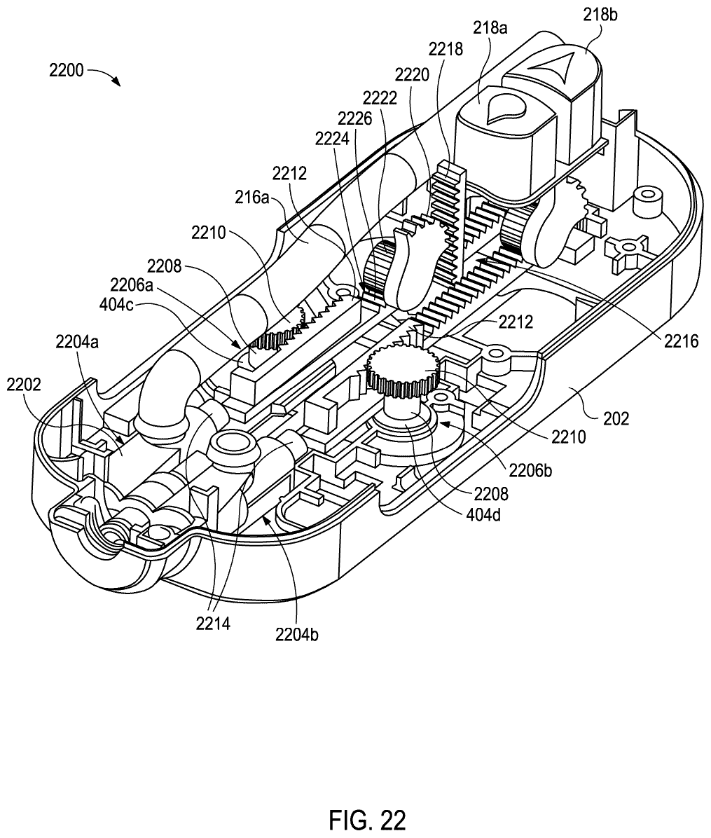

FIG. 22 is an exposed isometric view of another example flow control system 2200, according to one or more additional embodiments of the present disclosure. The flow control system 2200 may be similar in some respects to the flow control system 508 of FIGS. 5A-5B and therefore may be best understood with reference thereto. Similar to the flow control system 508, the flow control system 2200 may be arranged within the drive housing 202 and may form part of the suction irrigator 200 (FIGS. 2 and 4). Moreover, the flow control system 2200 includes a flow manifold 2202 that houses first and second internal valves 2204a,b, and the irrigation and suction buttons 218a,b are used to manually operate (actuate) the first and second internal valves 2204a,b, respectively.

Several component parts that may be otherwise contained within the drive housing 202, however, are not shown in FIGS. 22A-22B to enable discussion of the depicted component parts. For example, while the first conduit 216a (shown in dashed lines) is depicted, the second conduit 216b (FIGS. 5A-5B) and the tube connector 516 (FIGS. 5A-5B) are omitted in FIGS. 22A-22B, but would otherwise be included to facilitate the conveyance of fluids through the flow manifold 2202 and to (and from) the distal tip 226 (FIG. 2).

The flow control system 2200 includes first and second capstan assemblies 2206a,b that may be actuated (operated) to robotically operate the first and second internal valves 2204a,b, respectively. As illustrated, each capstan assembly 2206a,b includes a capstan shaft 2208 extending from or otherwise forming part of the third and fourth drive inputs 404c,d, respectively, such that actuation (rotation) of the third and fourth drive inputs 404c,d correspondingly rotates the capstan shaft 2208. A drive gear 2210 may be provided on or otherwise form part of each capstan shaft 2208, and the drive gear 2210 may be arranged to intermesh with a corresponding driven gear 2212. In the illustrated embodiment, the drive and driven gears 2210, 2212 comprise rack and pinion gears, where the drive gear 2210 comprises the pinion gear and the driven gear 2212 comprises the rack gear. In other embodiments, however, the drive and driven gears 2210, 2212 may comprise other types of gearing interfaces, such as a beveled gear interface, without departing from the scope of the disclosure.

In example operation to robotically actuate the first or second internal valve 2204a,b, the corresponding capstan assembly 2206a,b may be actuated to rotate the associated capstan shaft 2208. As the capstan shaft 2208 rotates, the drive gear 2210 drives against and moves the driven gear 2212 longitudinally (distally) within the drive housing 202 to actuate the corresponding internal valve 2204a,b. More specifically, in some embodiments, the driven gear 2212 may be driven distally until engaging a plunger 2214 forming part of the corresponding internal valve 2204a,b, and further distal movement of the driven gear 2212 against the plunger 2214 causes the corresponding internal valve 2204a,b to operate between closed and open positions (states).

The flow control system 2200 also includes a decoupling mechanism 2216 that allows manual actuation of the first and second internal valves 2204a,b without back driving the corresponding capstan assemblies 2206a,b. More specifically, and with reference to actuation of the first internal valve 2204a, the decoupling mechanism 2216 includes a rack gear 2218 extending from the irrigation button 218a and engageable with a driven gear 2220 rotatably mounted within the drive housing 202. As illustrated, the driven gear 2220 may include a pinion gear 2222 engageable with a drive shaft 2224 extending longitudinally within the drive housing 202. In some embodiments, for example the drive shaft 2224 may include or define a rack gear 2226, and the pinion gear 2222 may be engageable with the rack gear 2226 to drive the drive shaft 2224 longitudinally when the button 218a is manually depressed.

In at least one embodiment, as illustrated, the rack gear 2218 and the drive shaft 2224 may be oriented generally perpendicular to each other. Moreover, the drive shaft 2224 may extend parallel to the driven gear 2212. In some embodiments, as illustrated, the driven gear 2212 and the drive shaft 2224 may be arranged vertically adjacent to each other such that actuation (movement) of one of the rack gear 2218 or the drive shaft 2224 causes sliding engagement against the other. In other embodiments, however, the rack gear 2218 and the drive shaft 2224 may be vertically offset from each other. Moreover, a distal end of the drive shaft 2224 may be arranged adjacent to the first internal valve 2204a such that distal movement of the drive shaft 2224 will operate the first internal valve 2204a. More specifically, in some embodiments, the drive shaft 2224 may be driven distally until engaging the plunger 2214, and further distal movement of the drive shaft 2224 against the plunger 2214 causes the first internal valve 2204a to operate between closed and open positions (states).

To manually actuate the first internal valve 2204a, a user (e.g., a surgeon, bedside assist, etc.) may manually press down on the irrigation button 218a, which correspondingly drives the rack gear 2218 downward and against the driven gear 2220, thereby causing the driven gear 2220 to rotate. As the driven gear 2220 rotates, the pinion gear 2222 correspondingly rotates and drives against the drive shaft 2224, which drives the drive shaft 2224 distally and into engagement with the plunger 2014. This causes the plunger 2214 to move distally to actuate (operate) the first internal valve 2204b.

Since the drive shaft 2224 extends parallel to but does not drive against (engage) the driven gear 2212, the decoupling mechanism 2216 allows the irrigation button 218a to be manually depressed without acting on the capstan assembly 2206a. The same is true for manual actuation of the suction button 218b. Accordingly, the buttons 218a,b can be manually actuated (pressed) without back driving the third or fourth drive inputs 404c,d, or the motor used to actuate the drive inputs 404c,d.

Embodiments disclosed herein include:

A. A suction irrigator includes a drive housing having first and second drive inputs rotatably mounted thereto, a flow control system arranged within the drive housing and including a flow manifold that houses an internal valve, a button mounted to the drive housing and operatively coupled to the flow manifold such that manually depressing the button operates the internal valve between closed and open positions, a capstan shaft extending from the first drive input and including a drive gear, a driven gear operatively coupled to the button and selectively matable with the drive gear, wherein, when the drive and driven gears are engaged, actuation of the first drive input acts on the button and operates the internal valve, and an actuatable clutch plate operatively coupled to the second drive input such that selective actuation of the second drive input engages or disengages the drive and driven gears, wherein depressing the button when the drive and driven gears are disengaged fails to backdrive the first drive input or a motor operable to drive the first drive input.

B. A suction irrigator includes a drive housing, a flow control system arranged within the drive housing and including a flow manifold that houses an internal valve, a drive input rotatably mounted to the drive housing, a capstan assembly arranged within the drive housing and operatively coupled to the flow manifold such that rotation of the drive input actuates the capstan assembly and thereby operates the internal valve between closed and open positions, a button mounted to the drive housing, a drive shaft operatively coupled to the button such that manually depressing the button acts on the drive shaft to operate the internal valve between the closed and open positions, wherein the drive shaft is mechanically decoupled from the capstan assembly such that manual movement of the button fails to backdrive the drive input or a motor operable to drive the drive input.

C. A suction irrigator includes a drive housing, a flow control system arranged within the drive housing and including a flow manifold that houses an internal valve, a drive input rotatably mounted to the drive housing, a capstan assembly arranged within the drive housing and operatively coupled to the flow manifold such that rotation of the drive input actuates the capstan assembly and thereby operates the internal valve between closed and open positions, a button mounted to the drive housing and operatively coupled to the flow manifold such that manually depressing the button operates the internal valve between the closed and open positions, and a decoupling mechanism that mechanically decouples the button from the capstan assembly when the button is manually pressed such that manual movement of the button fails to backdrive the drive input or a motor operable to drive the drive input.

D. A method of operating a suction irrigator includes actuating a capstan assembly arranged within a drive housing of the suction irrigator, the suction irrigator including a flow control system arranged within the drive housing and including a flow manifold that houses an internal valve, a drive input rotatably mounted to the drive housing, wherein the capstan assembly is operatively coupled to the flow manifold and the drive input such that rotating the drive input actuates the capstan assembly, and a button mounted to the drive housing and operatively coupled to the flow manifold. The method further including operating the internal valve between closed and open positions as the capstan assembly is actuated, manually depressing the button and thereby operating the internal valve between the closed and open positions, and mechanically decoupling the button from the capstan assembly when the button is manually pressed with a decoupling mechanism, and thereby failing to backdrive the drive input or a motor that drives the drive input as the button is manually depressed.

Each of embodiments A, B, C, and D may have one or more of the following additional elements in any combination: Element 1: wherein the drive and driven gears comprise matable castellated features. Element 2: wherein the drive gear is rotatably mounted to the actuatable clutch plate and moves therewith. Element 3: further comprising a drive shaft extending from the second drive input and providing a helical profile matable with a profile slot defined in the clutch plate, wherein actuation of the second drive input rotates the drive shaft and thereby causes the helical profile to traverse profile slot, which raises or lowers the actuatable clutch plate. Element 4: further comprising an arm extending laterally from the button and defining a helical thread profile, and a helical gear extending from the driven gear and threaded into the helical thread profile, wherein, when the drive and driven gears are engaged, rotating the drive gear advances the helical gear into the helical thread profile and thereby acts on the button to operate the internal valve. Element 5: wherein the helical gear is secured vertically, but is rotatable relative to the arm to move the button vertically. Element 6: wherein, when the drive and driven gears are disengaged, manually depressing the button advances the helical gear into the helical thread profile as the button operates the internal valve between closed and open positions.

Element 7: wherein the capstan assembly includes a capstan shaft extending from the drive input and including a drive gear, and a driven gear mounted to the drive shaft and arranged to intermesh with the drive gear, the suction irrigator further comprising a drive member mounted to an end of the drive shaft and defining a helical slot, a follower pin extending from the drive shaft and received within the helical slot, wherein rotating the drive shaft either by actuation of the capstan assembly or manually depressing the button causes the follower pin to traverse the helical slot and drive the drive member into engagement with the internal valve to operate the internal valve. Element 8: further comprising a valve body forming part of the internal valve, wherein the drive member engages and drives the valve body to operate the internal valve. Element 9: further comprising a pinion gear provided on the drive shaft, and a rack gear extending from the button and engageable with the pinion gear such that manually depressing the button drives the rack gear against the pinion gear and thereby rotates the drive shaft. Element 10: wherein the drive and driven gears comprise matable helical gears. Element 11: further comprising a plunger forming part of the internal valve and defining a camming surface, a drive member mounted to an end of the drive shaft and engageable with the camming surface, a pinion gear provided on the drive shaft, and a rack gear extending from the button and engageable with the pinion gear, wherein manually depressing the button drives the rack gear against the pinion gear and rotates the drive shaft, which drives the drive member into engagement with the camming surface to operate the internal valve. Element 12: wherein the capstan assembly includes a cam driver extending from the drive input and providing a camming lobe engageable with the camming surface such that actuation of the capstan assembly drives the camming lobe into engagement with the camming surface and thereby operates the internal valve. Element 13: wherein the capstan assembly includes a capstan shaft extending from the drive input and including a drive gear, a driven gear mounted to the drive shaft and arranged to intermesh with the drive gear such rotating the drive gear causes the drive shaft to rotate, and a decoupling mechanism including an arcuate channel defined in the drive shaft, and a projection extending from the driven gear and receivable within the arcuate cutout, wherein the arcuate channel exhibits an angular magnitude that allows the drive shaft to rotate relative to the driven gear when the button is manually depressed. Element 14: wherein the drive and driven gears comprise matable bevel gears or matable helical gears. Element 15: wherein the drive member is cylindrical and exhibits a circular cross-section, and wherein the drive member is coupled to the drive shaft offset from a longitudinal axis of the drive shaft. Element 16: wherein the drive member exhibits an ovoid cross-sectional shape. Element 17: The suction irrigator of claim 28, wherein the capstan assembly includes a capstan shaft extending from the drive inputs and including a drive gear, and a driven gear arranged to intermesh with the drive gear such that rotation of the drive gear drives the driven gear longitudinally and into engagement with the internal valve to operate the internal valve. Element 18: wherein the driven gear is a first driven gear and the suction irrigator further comprises a rack gear extending from the button, and a second driven gear in engagement with the drive shaft and operatively coupled to the rack gear such that manually depressing the button drives the second driven gear against the drive shaft and thereby moves the drive shaft longitudinally and into engagement with the internal valve to operate the internal valve. Element 19: wherein the rack gear comprises a first rack gear and the second driven gear includes a pinion gear engageable with a second rack gear defined on the drive shaft. Element 20: wherein the drive gear extends parallel to the drive shaft, and wherein the drive gear and the drive shaft slidably engage each other.

Element 21: wherein the capstan assembly includes a capstan shaft extending from the drive input and including a drive gear, a driven gear arranged to intermesh with the drive gear and including a spur gear, and a ring mounted to the button within an annular recess defined on the button, the ring including a rack gear arranged to intermesh with the spur gear such that actuation of the capstan assembly drives the rack gear against a lower shoulder of the annular recess and thereby moves the button and operates the internal valve, wherein the decoupling mechanism includes the ring, which travels within the annular recess along a height of the annular recess when the button is manually depressed such that the ring fails to engage an upper shoulder of the button and, therefore, fails to drive the rack gear against the spur gear. Element 22: wherein the capstan assembly includes a capstan shaft extending from the drive input and defining a cam profile, a cam lifter operatively coupled to the button such that vertical movement of the button correspondingly moves the cam lifter in the same direction, a cam driver extended through the cam lifter and into the cam profile to operatively couple the cam lifter to the capstan shaft, a first lateral arm extending from the capstan shaft and engageable with a plunger forming part of the internal valve such that rotating the capstan shaft drives the first lateral arm against the plunger to operate the internal valve, and a second lateral arm extending from the cam lifter and engageable with the plunger, wherein the decoupling mechanism includes a combination of the cam driver and the cam profile such that manually depressing the button causes the cam driver to traverse the cam profile and rotate the cam lifter relative to the capstan shaft, which remains stationary, and wherein rotating the cam lifter drives the second lateral arm against the plunger to operate the internal valve. Element 23: wherein the cam profile comprises an angled slot defined in a sidewall of the capstan shaft. Element 24: wherein the cam lifter includes an enlarged head received within a slot defined in the button to operatively couple the cam lifter to the button. Element 25: wherein the capstan assembly includes a capstan shaft extending from the drive input and providing a cam surface, a rocker arm pivotably mounted to the drive housing at one or more lateral pins and pivotable about a pivot axis extending through the one or more lateral pins, the rocker arm including an engagement feature engageable against the cam surface such that rotation of the capstan shaft causes the rocker arm to pivot about the pivot axis, and an arm pivotably attached to the button such that pivoting movement of the rocker arm correspondingly moves the button and operates the internal valve, wherein manually depressing the button causes the rocker arm to pivot about the pivot axis, and wherein the decoupling mechanism includes the rocker arm and the engagement feature, which disengages from the cam surface when the rocker arm pivots as the button is manually depressed. Element 26: wherein the engagement feature comprises a pin extending between the rocker arm and the capstan shaft. Element 27: wherein the arm terminates in a boss pivotably received within a slot defined in the button. Element 28: wherein the capstan assembly includes a capstan shaft extending from the drive input and including a drive gear, a driven gear arranged to intermesh with the drive gear, a cam cylinder extending from the driven gear and defining an angled slot and a vertical slot contiguous with the angled slot, the cam cylinder extending about a portion of a valve body of the internal valve, and the button being operatively coupled to the valve body, and a follower pin receivable within the angled and vertical slots, wherein rotating the driven gear rotates the cam cylinder and causes the follower pin to traverse the angled slot and thereby draw the valve body downward to operate the internal valve, and wherein the decoupling mechanism includes a combination of the follower pin and the vertical slot such that manually depressing the button causes the follower pin to traverse the vertical slot without engaging the angled slot, and thereby without acting on the capstan assembly. Element 29: wherein the follower pin extends from an interior portion of the button and extends laterally inward to be received within the angled and vertical slots. Element 30: wherein the capstan assembly includes a capstan shaft extending from the drive input, a lateral arm extending from the capstan shaft such that rotation of the capstan shaft rotates the lateral arm, a crank slider arranged within the drive housing and operatively coupled to the lateral arm such that rotation of the lateral arm laterally shifts the crank slider, and an engagement member extending from the crank slider and engageable with a plunger forming part of the internal valve such that shifting the crank slider moves the engagement member against the plunger and thereby operates the internal valve, wherein the decoupling mechanism includes an angled surface provided on the capstan shaft, a follower pin extending from the button and engageable against the angled surface such that manually pressing the button causes the capstan shaft to rotate, and an arcuate channel defined on the drive input to receive the lateral arm, the arcuate channel exhibiting an angular magnitude that allows the lateral arm to rotate relative to the drive input when the button is manually depressed. Element 31: wherein the capstan assembly includes a shifting plate that provides a lateral arm in operable engagement with a valve body of the internal valve such that actuation of the capstan assembly drives against the valve body and operates the internal valve, wherein a portion of the button interposes the lateral arm and the valve body such that actuating the capstan assembly correspondingly moves the button, and wherein the decoupling mechanism includes the lateral arm being arranged above the portion of the button such that manually pressing the button acts on the valve body independent of the lateral arm. Element 32: wherein the capstan assembly includes a capstan shaft extending from the drive input and including a drive gear, an actuation wedge arranged within the drive housing and providing a driven gear arranged to intermesh with the driven gear, the actuation wedge providing a first angled engagement surface, and a second angled engagement surface provided on the button and engageable with the first angled engagement surface such that actuation of the capstan assembly moves the actuation wedge such that the first and second angled engagement surfaces slidably engage each other and the button is thereby moved to operate the internal valve, wherein the decoupling mechanism includes the first and second angled engagement surfaces such that manually pressing the button operates the internal valve while disengaging the second angled engagement surface from the first angled engagement surface without acting on the actuation wedge. Element 33: wherein the actuation wedge comprises a U-shaped structure that extends at least partially about an exterior of the button. Element 34: wherein the second angled engagement surface is defined on a wedge member provided on the button. Element 35: wherein the capstan assembly includes a capstan shaft extending from the drive input and including a drive gear, and a driven gear arranged to intermesh with the drive gear and engageable with a plunger forming part of the internal valve such that actuation of the capstan shaft drives the driven gear against the plunger to operate the internal valve, wherein the decoupling mechanism includes a first angled engagement surface provided on the plunger, and a second angled engagement surface provided on the button and slidably engageable with the first angled engagement surface when the button is manually pressed, and wherein manually pressing the button operates the internal valve while disengaging the driven gear from the plunger without acting the capstan assembly.

Element 36: wherein the capstan assembly includes a capstan shaft extending from the drive input and including a drive gear, a driven gear arranged to intermesh with the drive gear and including a spur gear, and a ring mounted to the button within an annular recess defined on the button, the ring including a rack gear arranged to intermesh with the spur gear, and wherein actuating the capstan assembly comprises driving the rack gear against a lower shoulder of the annular recess and thereby moving the button and operating the internal valve, and wherein the decoupling mechanism includes the ring and manually depressing the button comprises moving the ring within the annular recess along a height of the annular recess and without engaging an upper shoulder of the button, and failing to drive the rack gear against the spur gear as the button is manually depressed. Element 37: wherein the capstan assembly includes a capstan shaft extending from the drive input and providing a cam surface, and a rocker arm pivotably mounted to the drive housing at one or more lateral pins and pivotable about a pivot axis extending through the one or more lateral pins, and wherein actuating the capstan assembly comprises engaging an engagement feature of the capstan assembly against the cam surface such that rotation of the capstan shaft causes the rocker arm to pivot about the pivot axis, and moving the button and operating the internal valve as the rocker arm pivots, and wherein manually depressing the button comprises, pivoting the rocker arm about the pivot axis, and disengaging the engagement feature from the cam surface when the rocker arm pivots as the button is manually depressed, the decoupling mechanism including the rocker arm and the engagement feature. Element 38: wherein the capstan assembly includes a capstan shaft extending from the drive input and including a drive gear, a driven gear arranged to intermesh with the drive gear and engageable with a plunger forming part of the internal valve, and wherein actuating the capstan assembly comprises driving the driven gear against the plunger to operate the internal valve, wherein the decoupling mechanism includes a first angled engagement surface provided on the plunger, and a second angled engagement surface provided on the button, and wherein manually depressing the button comprises slidably engaging the second angled engagement surface against the first angled engagement surface and thereby operating the internal valve, and disengaging the driven gear from the plunger without acting the capstan assembly as the second angled engagement surface is driven against the first angled engagement surface.

By way of non-limiting example, exemplary combinations applicable to A, B, C, and D include: Element 4 with Element 5; Element 4 with Element 6; Element 7 with Element 8; Element 7 with Element 8; Element 7 with Element 10; Element 11 with Element 12; Element 11 with Element 13; Element 13 with Element 14; Element 11 with Element 15; Element 11 with Element 16; Element 17 with Element 18; Element 17 with Element 19; Element 17 with Element 20, Element 22 with Element 23; Element 22 with Element 24; Element 25 with Element 26; Element 25 with Element 27; Element 28 with Element 29; Element 32 with Element 33; and Element 32 with Element 34.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A suction irrigator, comprising:
a drive housing;
a flow control system arranged within the drive housing and including a flow manifold that houses an internal valve;
a drive input rotatably mounted to the drive housing;
a capstan assembly arranged within the drive housing and operatively coupled to the flow manifold such that rotation of the drive input actuates the capstan assembly and thereby operates the internal valve between closed and open positions;
a button mounted to the drive housing and operatively coupled to the flow manifold such that manually depressing the button operates the internal valve between the closed and open positions; and
a decoupling mechanism that mechanically decouples the button from the capstan assembly when the button is manually pressed such that manual movement of the button fails to backdrive the drive input or a motor operable to drive the drive input.

2. The suction irrigator of claim 1, wherein the capstan assembly includes:
a capstan shaft extending from the drive input and including a drive gear;
a driven gear arranged to intermesh with the drive gear and including a spur gear; and
a ring mounted to the button within an annular recess defined on the button, the ring including a rack gear arranged to intermesh with the spur gear such that actuation of the capstan assembly drives the rack gear against a lower shoulder of the annular recess and thereby moves the button and operates the internal valve,
wherein the decoupling mechanism includes the ring, which travels within the annular recess along a height of the annular recess when the button is manually depressed such that the ring fails to engage an upper shoulder of the button and, therefore, fails to drive the rack gear against the spur gear.

3. The suction irrigator of claim 1, wherein the capstan assembly includes:
a capstan shaft extending from the drive input and defining a cam profile;
a cam lifter operatively coupled to the button such that vertical movement of the button correspondingly moves the cam lifter in the same direction;
a cam driver extended through the cam lifter and into the cam profile to operatively couple the cam lifter to the capstan shaft;
a first lateral arm extending from the capstan shaft and engageable with a plunger forming part of the internal valve such that rotating the capstan shaft drives the first lateral arm against the plunger to operate the internal valve; and
a second lateral arm extending from the cam lifter and engageable with the plunger,
wherein the decoupling mechanism includes a combination of the cam driver and the cam profile such that manually depressing the button causes the cam driver to traverse the cam profile and rotate the cam lifter relative to the capstan shaft, which remains stationary, and
wherein rotating the cam lifter drives the second lateral arm against the plunger to operate the internal valve.

4. The suction irrigator of claim 3, wherein the cam profile comprises an angled slot defined in a sidewall of the capstan shaft.

5. The suction irrigator of claim 3, wherein the cam lifter includes an enlarged head received within a slot defined in the button to operatively couple the cam lifter to the button.

6. The suction irrigator of claim 1, wherein the capstan assembly includes:
a capstan shaft extending from the drive input and providing a cam surface;
a rocker arm pivotably mounted to the drive housing at one or more lateral pins and pivotable about a pivot axis extending through the one or more lateral pins, the rocker arm including:
an engagement feature engageable against the cam surface such that rotation of the capstan shaft causes the rocker arm to pivot about the pivot axis; and
an arm pivotably attached to the button such that pivoting movement of the rocker arm correspondingly moves the button and operates the internal valve,
wherein manually depressing the button causes the rocker arm to pivot about the pivot axis, and
wherein the decoupling mechanism includes the rocker arm and the engagement feature, which disengages from the cam surface when the rocker arm pivots as the button is manually depressed.

7. The suction irrigator of claim 6, wherein the engagement feature comprises a pin extending between the rocker arm and the capstan shaft.

8. The suction irrigator of claim 6, wherein the arm terminates in a boss pivotably received within a slot defined in the button.

9. The suction irrigator of claim 1, wherein the capstan assembly includes:
a capstan shaft extending from the drive input and including a drive gear;
a driven gear arranged to intermesh with the drive gear;
a cam cylinder extending from the driven gear and defining an angled slot and a vertical slot contiguous with the angled slot, the cam cylinder extending about a portion of a valve body of the internal valve, and the button being operatively coupled to the valve body; and
a follower pin receivable within the angled and vertical slots,
wherein rotating the driven gear rotates the cam cylinder and causes the follower pin to traverse the angled slot and thereby draw the valve body downward to operate the internal valve, and
wherein the decoupling mechanism includes a combination of the follower pin and the vertical slot such that manually depressing the button causes the follower pin to traverse the vertical slot without engaging the angled slot, and thereby without acting on the capstan assembly.

10. The suction irrigator of claim 9, wherein the follower pin extends from an interior portion of the button and extends laterally inward to be received within the angled and vertical slots.

11. The suction irrigator of claim 1, wherein the capstan assembly includes:
a capstan shaft extending from the drive input;
a lateral arm extending from the capstan shaft such that rotation of the capstan shaft rotates the lateral arm;
a crank slider arranged within the drive housing and operatively coupled to the lateral arm such that rotation of the lateral arm laterally shifts the crank slider; and
an engagement member extending from the crank slider and engageable with a plunger forming part of the internal valve such that shifting the crank slider moves the engagement member against the plunger and thereby operates the internal valve,
wherein the decoupling mechanism includes:
an angled surface provided on the capstan shaft;
a follower pin extending from the button and engageable against the angled surface such that manually pressing the button causes the capstan shaft to rotate; and
an arcuate channel defined on the drive input to receive the lateral arm, the arcuate channel exhibiting an angular magnitude that allows the lateral arm to rotate relative to the drive input when the button is manually depressed.

12. The suction irrigator of claim 1, wherein the capstan assembly includes a shifting plate that provides a lateral arm in operable engagement with a valve body of the internal valve such that actuation of the capstan assembly drives against the valve body and operates the internal valve,
wherein a portion of the button interposes the lateral arm and the valve body such that actuating the capstan assembly correspondingly moves the button, and
wherein the decoupling mechanism includes the lateral arm being arranged above the portion of the button such that manually pressing the button acts on the valve body independent of the lateral arm.

13. The suction irrigator of claim 1, wherein the capstan assembly includes:
a capstan shaft extending from the drive input and including a drive gear;
an actuation wedge arranged within the drive housing and providing a driven gear arranged to intermesh with the driven gear, the actuation wedge providing a first angled engagement surface; and
a second angled engagement surface provided on the button and engageable with the first angled engagement surface such that actuation of the capstan assembly moves the actuation wedge such that the first and second angled engagement surfaces slidably engage each other and the button is thereby moved to operate the internal valve,
wherein the decoupling mechanism includes the first and second angled engagement surfaces such that manually pressing the button operates the internal valve while disengaging the second angled engagement surface from the first angled engagement surface without acting on the actuation wedge.

14. The suction irrigator of claim 13, wherein the actuation wedge comprises a U-shaped structure that extends at least partially about an exterior of the button.

15. The suction irrigator of claim 13, wherein the second angled engagement surface is defined on a wedge member provided on the button.

16. The suction irrigator of claim 1, wherein the capstan assembly includes:
a capstan shaft extending from the drive input and including a drive gear; and
a driven gear arranged to intermesh with the drive gear and engageable with a plunger forming part of the internal valve such that actuation of the capstan shaft drives the driven gear against the plunger to operate the internal valve,
wherein the decoupling mechanism includes:
a first angled engagement surface provided on the plunger; and
a second angled engagement surface provided on the button and slidably engageable with the first angled engagement surface when the button is manually pressed, and
wherein manually pressing the button operates the internal valve while disengaging the driven gear from the plunger without acting the capstan assembly.

17. A method of operating a suction irrigator, comprising:
actuating a capstan assembly arranged within a drive housing of the suction irrigator, the suction irrigator including:
a flow control system arranged within the drive housing and including a flow manifold that houses an internal valve;
a drive input rotatably mounted to the drive housing, wherein the capstan assembly is operatively coupled to the flow manifold and the drive input such that rotating the drive input actuates the capstan assembly; and
a button mounted to the drive housing and operatively coupled to the flow manifold;
operating the internal valve between closed and open positions as the capstan assembly is actuated;
manually depressing the button and thereby operating the internal valve between the closed and open positions; and
mechanically decoupling the button from the capstan assembly when the button is manually pressed with a decoupling mechanism, and thereby failing to backdrive the drive input or a motor that drives the drive input as the button is manually depressed.

18. The method of claim 17, wherein the capstan assembly includes a capstan shaft extending from the drive input and including a drive gear, a driven gear arranged to intermesh with the drive gear and including a spur gear, and a ring mounted to the button within an annular recess defined on the button, the ring including a rack gear arranged to intermesh with the spur gear, and wherein actuating the capstan assembly comprises:
driving the rack gear against a lower shoulder of the annular recess and thereby moving the button and operating the internal valve, and
wherein the decoupling mechanism includes the ring and manually depressing the button comprises:
moving the ring within the annular recess along a height of the annular recess and without engaging an upper shoulder of the button; and
failing to drive the rack gear against the spur gear as the button is manually depressed.

19. The method of claim 17, wherein the capstan assembly includes a capstan shaft extending from the drive input and providing a cam surface, and a rocker arm pivotably mounted to the drive housing at one or more lateral pins and pivotable about a pivot axis extending through the one or more lateral pins, and wherein actuating the capstan assembly comprises:
engaging an engagement feature of the capstan assembly against the cam surface such that rotation of the capstan shaft causes the rocker arm to pivot about the pivot axis; and
moving the button and operating the internal valve as the rocker arm pivots, and
wherein manually depressing the button comprises:
pivoting the rocker arm about the pivot axis; and
disengaging the engagement feature from the cam surface when the rocker arm pivots as the button is manually depressed, the decoupling mechanism including the rocker arm and the engagement feature.

20. The method of claim 17, wherein the capstan assembly includes a capstan shaft extending from the drive input and including a drive gear, a driven gear arranged to intermesh with the drive gear and engageable with a plunger forming part of the internal valve, and wherein actuating the capstan assembly comprises:
driving the driven gear against the plunger to operate the internal valve,
wherein the decoupling mechanism includes a first angled engagement surface provided on the plunger, and a second angled engagement surface provided on the button, and wherein manually depressing the button comprises:
slidably engaging the second angled engagement surface against the first angled engagement surface and thereby operating the internal valve; and
disengaging the driven gear from the plunger without acting the capstan assembly as the second angled engagement surface is driven against the first angled engagement surface.
